(19) United States
(12) Patent Application Publication
(10) Pub. No.: US 2014/0088553 A1
(43) Pub. Date: Mar. 27, 2014

(54) RETRACTABLE PLUNGER DESIGN FOR INJECTION CONTROL DEVICE FOR PROPORTIONAL INJECTION EXTRACTION DURING THE SYRINGE'S INSERTION EXTRACTION

(71) Applicant: Hugh Hetherington, Bozeman, MT (US)

(72) Inventor: Hugh Hetherington, Bozeman, MT (US)

(21) Appl. No.: 14/025,933

(22) Filed: Sep. 13, 2013

Related U.S. Application Data
(63) Continuation-in-part of application No. 13/734,974, filed on Jan. 5, 2013.
Application No. 13/734,974 is a continuation-in-part of application No. 12/285,203, filed on Sep. 30, 2008.
Application No. 12/285,203 is a continuation-in-part of application No. 12/078,603, filed on Apr. 2, 2008, now Pat. No. 8,133,208.

(51) Int. Cl. A61M 5/315; A61M 5/20

(52) U.S. Cl. CPC A61M 5/31585; A61M 5/20
USPC 604/506; 604/211; 604/131

(57) ABSTRACT

An injection control device (ICD) for a syringe, injects/extracts material into/from at a rate controllably proportional to the rate of movement of the syringe. A transmission reference member is extendable outward from the ICD body, being connected to a transmission system/clutch which has an engage/disengage trigger for independent movement of a plunger arm. The ICD, via the transmission translates motion from movement of the body, in relation to a fixed state of the transmission reference member, to action on the plunger of an accommodated syringe. The clutch also allows the transmission reference member to be positioned, without transferring power to the transmission system.

### CROSS REFERENCE TO RELATED APPLICATIONS

This application is a Continuation-In-Part of U.S. patent application Ser. No. 13/734,974 filed Jan. 5, 2013 which is a Continuation-In-Part of U.S. patent application Ser. No. 12/285,203 filed Sep. 30, 2008, which is a Continuation-In-Part of U.S. patent application Ser. No. 12/078,603, filed Apr. 2, 2008, now issued as U.S. Pat. No. 8,133,208 on Mar. 13, 2012, and claims benefit to the priorities thereof. The contents therein being incorporated herein by reference in their entirety.

### FIELD

This disclosure relates to an injection or extraction device, referred to hereafter as the injection control device (ICD). More particularly, this disclosure relates a retractable plunger design for a semi-automatic hand operable ICD that proportionally injects or extracts material while the syringe's cannula's is inserted or extracted.

### BACKGROUND

Injection or extraction of material, for example, a filler material or fat cells, etc., in a patient requires a significant level of skill, particularly in the cosmetic surgery industry where a measured amount of the material must be “evenly” injected or removed. Too little or too much displacement of material causes an unnatural appearance in the skin or other treated areas of the body. The traditional method is to manually withdraw or inject the cannula of the syringe while manually manipulating the syringe's plunger in synchronicity. Of course, it goes without saying this approach is sensitive to the practitioner's skill level and produces different results for different passes. Being subject to human error, inconsistent results (e.g., lumps, thin lines, voids, etc.) often occur, as well as possible damage to the patient. Accordingly, there has been a long-standing need in the discipline to devise systems and methods for addressing the problems discussed above.

### SUMMARY

The following presents a simplified summary in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

The foregoing needs are met, to a great extent, by the present disclosure, wherein methods and systems are provided wherein various embodiments permit a controlled metering of injection material into a patient/object and withdrawal of material from a patient/object.

In accordance with one aspect of the present disclosure, an injection control device (ICD) for a syringe, adapted to inject/extract material into/from a subject at a rate proportional to the rate of movement of the syringe is provided, comprising: an ICD body with a proximal and distal end, comprising: a syringe holder positioned at the proximal end, securing at least one accommodated syringe from movement relative to the body; a reference member opening at the proximal end; and an arm opening along the body, to accommodate travel of an exposed plunger retraction arm of a plunger of the at least one accommodated syringe; a transmission reference member extendable outward from the body via the reference member opening; a clutch assembly coupled to the body with an engagement/disengagement release trigger to allow independent movement of the plunger retraction arm; and a transmission system coupled to the body, controllably engaged to the clutch, wherein a first power transfer section of the transmission is connected to the transmission reference member and a second power transfer section of the transmission is connected to a plunger of the at least one accommodated syringe, the transmission system configured to translate motion from movement of the body, in relation to a fixed state of the transmission reference member, to action on the plunger of the at least one accommodated syringe, wherein the clutch is configured to allow the transmission reference member to be positioned, without transferring power to the transmission system, wherein the plunger action is proportional to the movement of the body, resulting in material being injected/extracted into/from a subject as a cannula of the at least one accommodated syringe is traveling with the movement of the body.

In accordance with another aspect of the present disclosure, an injection control device (ICD) for a syringe, adapted to inject/extract material into/from a subject at a rate proportional to the rate of movement of the syringe is provided, comprising: an ICD body with a proximal and distal end, comprising: a syringe holder positioned at the proximal end, securing the at least one accommodated syringe from movement relative to the body; and means for accommodating travel of an exposed plunger retraction arm of a plunger of the at least one accommodated syringe; a reference member extendable outward from the body via the reference member opening; a means for clutching coupled to the body with a triggering means to allow independent movement of the plunger retraction arm; and a means for redirecting motion coupled to the body, controllably engaged to the means for clutching, wherein a first power transfer section of the means for redirecting motion is connected to the reference member and a second power transfer section of the means for redirecting motion is connected to a plunger of the at least one accommodated syringe, the means for redirecting motion configured to translate motion from movement of the body, in relation to a fixed state of the reference member, to action on the plunger of the at least one accommodated syringe, wherein the means for clutching is configured to allow the reference member to be positioned, without transferring power to the means for redirecting motion, wherein the plunger action is proportional to the movement of the body, resulting in material being injected/extracted into/from a subject as a cannula of the at least one accommodated syringe is traveling with the movement of the body.

In accordance with another aspect of the present disclosure, a device as described above is provided, wherein the ICD body is a multi-piece body and the arm opening delineates a section of the body that is retractable, exposing the at least one accommodated syringe; and/or the second power transfer section of the transmission is indirectly coupled to the plunger of the at least one accommodated syringe; and/or the cannula of the at least one accommodated syringe is at least one of a plurality of cannulas and a flexible cannula; and/or further comprising a syringe, wherein the syringe contains at least one of fat, stem cells, hyaluronic acid, polymethylmethacrylate, hydroxyapatite, drug, vaccine, botulinum toxin, bone cement, demineralized bone, and hydrogel; and/or the transmission reference member is at least one of an optical or laser ranging, electromagnetic ranging, and stereotactic system utilizing a computer-controlled servo to control the plunger.

In accordance with yet another aspect of the present disclosure, a method of injecting/extracting material into/from a subject at a rate controllably proportional to the rate of movement of a syringe attached to an injection control device (ICD) is provided, the device comprising: placing an ICD with an at least one accommodated syringe's cannula upon or into a subject's tissue, wherein the ICD comprises: an ICD body with a proximal and distal end, comprising: a syringe holder positioned at the proximal end, securing the at least one accommodated syringe from movement relative to the body; a reference member opening at the proximal end; and an arm opening along the body, to accommodate travel of an exposed plunger retraction arm of a plunger of the at least one accommodated syringe; a transmission reference member extendable outward from the body via the reference member opening; a clutch assembly coupled to the body with an engagement/disengagement release trigger to allow independent movement of the plunger retraction arm; and a transmission system coupled to the body, controllably engaged to the clutch, wherein a first power transfer section of the transmission is connected to the transmission reference member and a second power transfer section of the transmission is connected to a plunger of the at least one accommodated syringe, the transmission system configured to translate motion from movement of the body, in relation to a fixed state of the transmission reference member, to action on the plunger of the at least one accommodated syringe, wherein the clutch is configured to allow the transmission reference member to be positioned, without transferring power to the transmission system, positioning the transmission reference member; and pressing on the transmission reference member and withdrawing or advancing the body of the ICD, wherein the plunger action is proportional to the movement of the body, resulting in material being injected/extracted into/from a subject as the at least one accommodated syringe's cannula is traveling with the movement of the body.

In accordance with yet another aspect of the present disclosure, a method as described above is provided, wherein the release trigger and clutch is a mechanism comprising: a translatable axle of the transmission system having a first diameter and a second diameter smaller than the first diameter, an end thereof being directly or indirectly engaged via the release trigger; an axle gear attached to the translatable axle, a teeth of the axle gear contacting a teeth of a main gear of the transmission system, in both an engaged and disengaged state of the release trigger; and a plunger gear displaced from the axle gear and in alignment with the translatable axle, a center hole of the plunger gear sized larger than the second diameter of the translatable axle and smaller than the first diameter of the translatable axle, so as to cause engagement of the plunger gear with the translatable axle when the first diameter of the translatable axle is translated into the plunger gear hole and to cause disengagement of the plunger gear when the second diameter of the translatable axle is translated into the plunger gear hole, wherein in an disengaged state, the plunger gear is disengaged from the clutch and transmission system, allowing the plunger to be moved without affecting the first power transfer section of the transmission system; and/or the release trigger is in an disengaged state; and/or the cannula of the at least one accommodated syringe is at least one of a plurality of cannulas and a flexible cannula, and the device is used for at least one of a endoscopic, endovascular and laparoscopic procedure.

### BRIEF DESCRIPTION OF THE DRAWINGS

FIG. 1 is an illustration of a side view of an embodiment of an injection control device according to a first configuration.

FIG. 2 is an illustration of a side view of a separated embodiment of the injection control device of FIG. 1

FIG. 3 is an illustration of a cut-away view of the embodiment of the injection control device of FIG. 1.

FIG. 4 is a close-up reverse illustration of the interior of an embodiment of an injection control device.

FIG. 5 is a bottom-side illustration of an embodiment of the injection control device with the syringe rack removed from view.

FIG. 6 is a perspective view illustration of the syringe rack arrangement of an embodiment of the injection control device.

FIG. 7 is an illustration of an embodiment of an injection control device with multiple gears.

FIG. 8 is an illustration of a perspective bodiless view of a “rackless” embodiment of an injection control device according to a second configuration.

FIG. 9 is an illustration of a perspective bodiless view of another “rackless” embodiment of an injection control device according to a third configuration.

FIG. 10 is an illustration of a bodiless cut-away view of an embodiment of the injection control device of FIG. 9.

FIG. 11 is an illustration of a “bodied” cut-away view of the embodiment of injection control device of FIG. 9 with adjustable stops.

FIG. 12 is an illustration of a perspective view of an embodiment of an injection control device according to a fourth configuration.

FIG. 13 is an illustration of a perspective view of an embodiment of an injection control device according to a fifth configuration.

FIG. 14 is an illustration of a cut-away view of the embodiment of the injection control device of FIG. 13.

FIG. 15 is an illustration of a cut-away view of the embodiment of the injection control device of FIG. 13 but in an “extended” position.

FIG. 16 is an illustration of a perspective view of an embodiment of an injection control device according to a sixth configuration.

FIG. 17 is an illustration of a cut-away view of the embodiment of the injection control device of FIG. 16.

FIG. 18 is an illustration of a cut-away view of the embodiment of the injection control device of FIG. 16 but in an “extended” position.

FIG. 19 is an illustration of a bodiless cut-away view of the embodiment of the injection control device of FIG. 18, showing the plunger activating gear train.

FIG. 20 is an illustration of a bodiless cut-away view of the embodiment of the injection control device of FIG. 19, showing the plunger positioning guide gear train.

FIG. 21 is an illustration of a syringe adapter for use with some embodiments of the injection control devices.

FIG. 22 is an illustration of a multi-cannula syringe for use some embodiments of the injection control devices.

FIG. 23 is an illustration of a flexible-cannula syringe for use with some embodiments of the injection control device.

FIG. 24 is an illustration of a cut-away view of a modified embodiment of an injection control device with a disengagable positioning guide with an associated blowup view in FIG. 24A.

FIG. 25 is an illustration of a perspective view of the main body of a modified embodiment of an injection control device configured for independent plunger movement, with the positioning guide removed from view.

FIG. 26 is another illustration of the embodiment of FIG. 25, but with the finger grip section removed.

FIG. 27 is a top view illustration of a clutch assembly in a engaged position.

FIG. 28A is an illustration of the clutch button engaged with the clutch assembly of FIG. 27.

FIG. 28B is an illustration of the clutch button pushed “out of” body of the clutch assembly of FIG. 27.

FIG. 29 is an illustration of a multi-syringe device.

### DETAILED DESCRIPTION OF THE DRAWINGS

The claimed subject matter is now described with reference to the drawings, wherein like reference numerals are used to refer to like elements throughout. In the following description, for purposes of explanation, numerous specific details are set forth in order to provide a thorough understanding of the claimed subject matter. It may be evident, however, that such subject matter may be practiced without these specific details.

Many different filler materials have been used for tissue augmentation or treatment, including live cells from the patient. When injecting the material the practitioner must avoid “clumping” as he withdraws/insets the cannula (or conversely, under-injecting). When extracting material (for example, fat cells from the patient), the practitioner must exercise equal care to avoid removing too many fat cells lest a depression form on the patients dermis where the cells have been removed.

In any scenario, the practitioner must exercise extreme care to coordinate the movement of the cannula with the movement of the syringe's plunger. Also, for multiple passes in the same area, the practitioner is tasked with repeating the exact amount delivered (withdrawn) per pass.

If fat cells are utilized, they are known to be fragile and the augmentation may be temporary if a significant proportion of the fat cells die. To maximize the survival of injected fat cells, the fat cells must be evenly distributed through the recipient tissue in small parcels. The parcels must be small enough that they can obtain adequate nutrition through plasmatic imbibition until such time as neovascularization of the fat parcels occurs. To accomplish this, the cannula is passed through the tissue multiple times, depositing a small amount of fat with each pass.

The conventional method of injecting fat and other materials is to manually advance the plunger into the syringe as the cannula is withdrawn from the tissue. The key to maximizing survival of the grafted fat is to make many passes. An insufficient number of passes will result in resorption of a portion of the fat cells. An excessive number of passes results in prolonged swelling of the tissue often taking several months to resolve. The prolonged swelling and variable results discourages the use of facial fat grafting. It is also difficult to manually gauge the amount of fat injected with each pass of the cannula.

In an attempt to address this difficulty, some practitioners have used a ratchet gun to inject the fat. However, the trigger mechanism associated with a ratchet gun injects a small amount of fat each time the trigger is squeezed. It essentially functions like a stationary caulking gun. This device allows the operator somewhat better control over the release of the fat into the tissue however, the amount of fat injected is not proportional with the distance that the cannula is passed through the tissue. Therefore, overly large amounts or overly small amounts of filler material or fat can be injected along the injection track. Thus, these attempts have not adequately addressed the problems inherent to traditional manual injection methods.

The exemplary devices and methods described herein provide effective solutions to difficulties of the prior art, wherein in various embodiments a controlled amount of material, such as, for example, a filler is automatically deposited with each pass of the cannula. In principal, the cannula is advanced into the tissue to create a tract or tunnel within the targeted area. Then, as the cannula is withdrawn, the material is uniformly deposited though the tract or tunnel via the automatic metering system. The automatic metering system incorporates a syringe activating mechanism coupled to a gearing system which proportions the deposition to the retraction of the cannula. Conversely, extraction of material can be similarly “metered” in a proportional manner, being drawn as the cannula is inserted into the subject, or even as the cannula is being withdrawn from the subject.

By use of the exemplary devices and methods described herein, more consistent and uniform distribution of the material injected can be achieved with less cannula passes as well as having less dependence on the skills of the individual surgeon. Additionally, it should be appreciated that though the exemplary embodiments described herein are described in the context of using fat as the filler material, other materials that may or may not be a filler, whether organic or non-organic, living or non-living, may be used without departing from the spirit and scope of this disclosure.

For example, the exemplary ICD can be used with living cells, non-limiting examples being fat, stem cells and so forth. Additionally, synthetic fillers may be used such as, hyaluronic acid (e.g., Restylane® registered by HA North American Sales AB, Juvederm® registered by Allergan, Inc. Irvine, Calif.), polymethylmethacrylate (e.g., Artefill® registered by Suneva Medical, Inc.), hydroxyapatite (e.g., Radiesse® registered by Merz Aesthetics, Inc.), and so forth. Drugs may also be administered by the exemplary ICD, as one example, the ICD in concert with the multiple needle hub could be used to inject chemotherapeutic agents into solid tumors, mesotherapy, sclerotherapy to treat varicose veins, surface treatment of implanted medical devices that are contaminated with a biofilm. Continuing, biologicals such as vaccines could be administered, as well as the botulinum toxin. Moreover, bone cement, demineralized bone, hydrogels and other substances could be used as the “material” in the exemplary ICD.

It should be also appreciated that, in addition to the benefits listed above, by minimizing the number of cannula passes in the tissue, less trauma is effectuated upon the tissue, resulting in less swelling in the patient's body. Moreover, by metering the amount of fat (filler material) in the injection areas, less filler material is necessary to achieve the desired results. These and other advantages will be made more evident in the forthcoming sections.

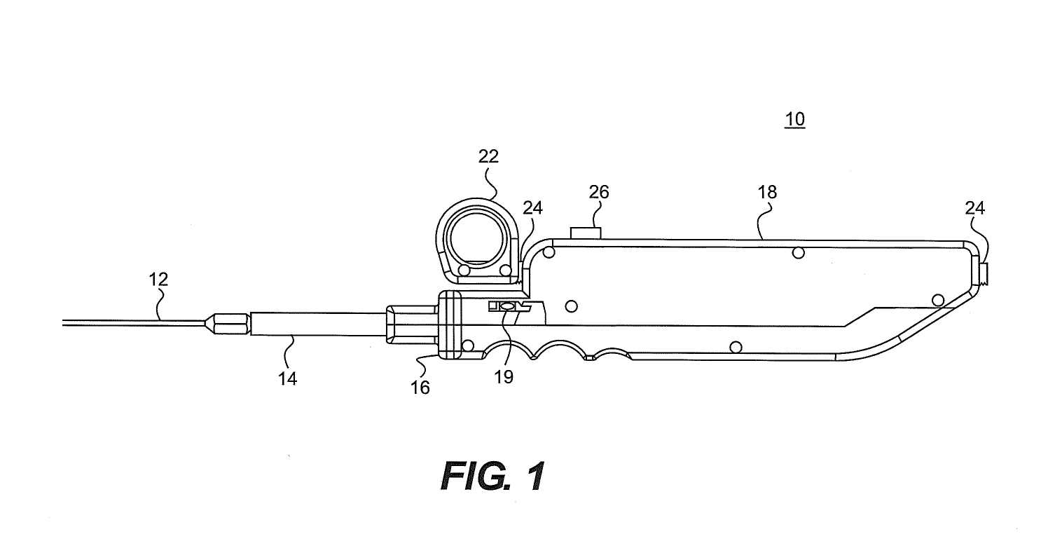

FIG. 1 is an illustration of a side view 10 of an exemplary injection control device according to an embodiment of the invention. The exemplary injection control device is illustrated with a cannula or needle 12 coupled to a cannula mating section 14. It should be apparent that the cannula 12 may be removable or be of a disposable form. The cannula mating section 14 may be referred to as the syringe of the exemplary injection control device. The syringe 14 may be configured to be supported and/or held securely by a syringe-supporting section 16 of the body 18, preventing movement of the syringe 14 relative to the body 18. The syringe 14 may also be disposable, if so desired, and may be configured in varying sizes, according to design or application preference. Accordingly, the syringe supporting section 16 may be configured to be adapted to various shapes or sizes of the syringe 14, according to design or application preference. While the cannula 12 is illustrated as having a straight shape, other curvatures or shapes may be used according to application preference.

The body 18 is illustrated as containing a latch 19 which operates to secure the upper and lower portions of the body 18, during assembly. The body 18 accommodates an exposed ring 22 which is connected to a positioning rack 24 (partially obscured) which is housed or protected by the body 18. The positioning rack 24 is shown in FIG. 1 as being situated to travel through the body 18 and is subject to engagement of the brake 26. In some embodiments, the positioning rack 24 may be placed exterior of the body 18, according to design preference, such as, for a non-limiting example, a sliding arrangement as seen in older slide rules. The brake 26 operates to prevent travel of the positioning rack 24 when engaged, or conversely, when dis-engaged, depending on design implementation.

While FIG. 1 illustrates the exposed ring 22 as being circular in shape, it should be understood that other shapes, closed or open, may be used without departing from the spirit and scope of this disclosure. In fact, in some embodiments, it may be desirable to have a “flat” surface or “plate” rather than the exposed ring 22, depending on the practitioner's preference or application.

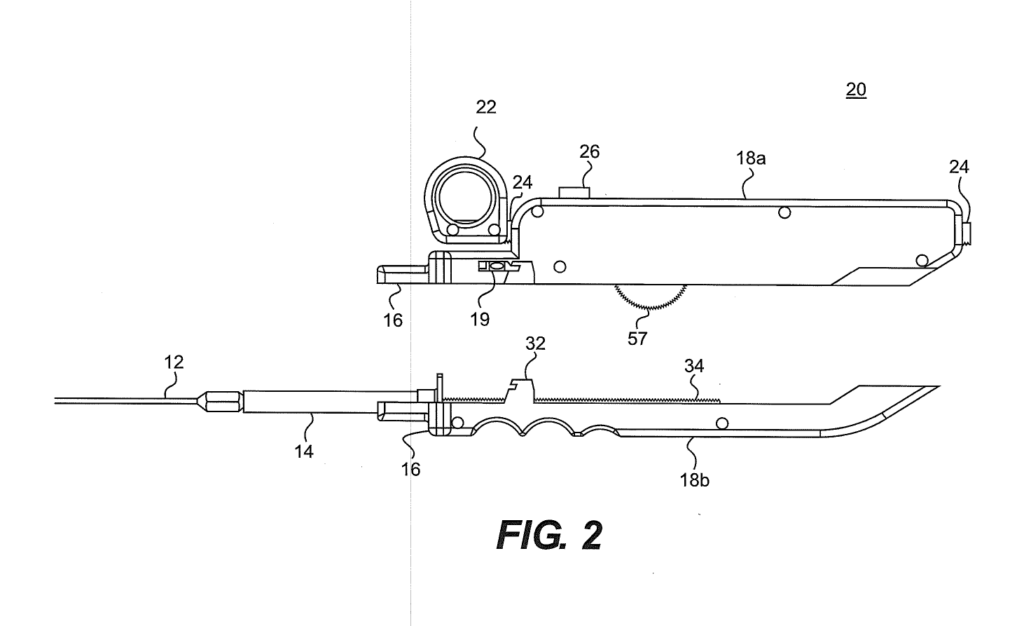

FIG. 2 is an illustration of a side view 20 of the exemplary injection control device of FIG. 1 with the upper body portion 18a and lower body portion 18b of the body 18 separated. Of note is the exposed latch engagement member 32 used for attachment to the latch 19 when the upper body portion 18a and lower body portion 18b are attached to each other. Also, FIG. 2 illustrates the lower portion of the exposed syringe rack gear 57 and the upper portion of the corresponding syringe rack 34. It should be appreciated that other forms of the latch engagement member 32 may be used than that shown in FIG. 2. That is, instead of latching with a slidable latch 19, a twisting or screwing, or otherwise engaging motion may be used with an appropriately designed latch engaging member 32, to achieve the desired securing operation, without departing from the spirit and scope of this disclosure. Therefore, other devices or mechanisms known in the art for securing the upper portion 18a and the lower portion 18b of the body 18 may be contemplated, according to design or efficiency preference.

Further, it should be appreciated that the exemplary embodiment shown in FIG. 2 may also be configured so that the body 18 is separated into a different configuration, such as to be arranged in “left” and/or “right” or other arrangements, as opposed to “upper” and/or “lower” etc. Therefore, it should be apparent that other shapes, whether paired or multiplied, or separation methodologies ranging from sliding, twisting, screwing, snapping, etc., for example, may be used to enable the practitioner to access the interior of the exemplary injection control device. It should also be appreciated that in some embodiments, a gripping portion may be provided on the surface of the body 18 to enable a practitioner a secure hold of the exemplary injection control device.

Additionally, while the exemplary injection control device is shown in FIG. 2 with a body 18 that may be separated, it is contemplated that a uni-body implementation may be used. That is, the body 18 may be formed as a single piece, not separable wherein the syringe 14 is “attached” to the body 18. Thus, a single body configuration may be made without departing from the spirit and scope of this subject matter.

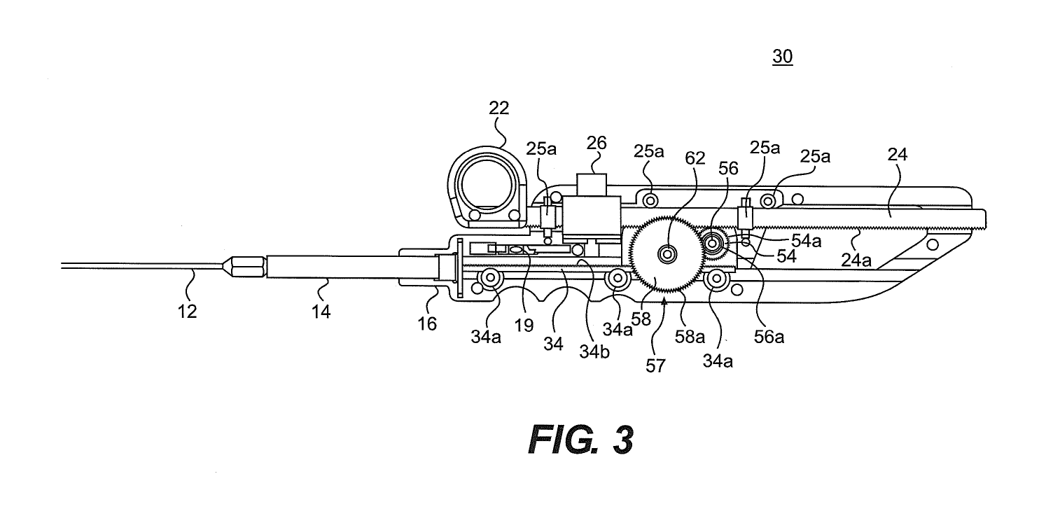

FIG. 3 is an illustration of an axial cut-away view 30 of the exemplary injection control device of FIG. 1. The cut-away view 30 reveals an exemplary gearing arrangement suitable for accomplishing at least one of the goals of the exemplary injection control device. For example, using the gearing arrangement shown in FIG. 3, it should be apparent to one of ordinary skill in the art that during the operation of the exemplary injection control device, as the ring 22 is fixed in place and the body of the injection control device is moved to the “right”, the syringe rack 34 will move to the “left” acting as a plunger into the syringe 14 being held in the syringe supporting section 16. Therefore, any filler material in the syringe 14 will be expelled into the cannula 12. Based on appropriate gearing ratios of the exemplary gearing arrangement, a very precise and controlled injection of the filler material can be accomplished in proportion to the travel of the cannula 12, resulting in a consistent amount of filler material being injected per travel distance of the cannula 12. This allows for uniform delivery of filler material (or other material) per unit area of the patient, thus providing the desired dose per “area”, with minimal technical expertise. Conversely, the reverse can be accomplished, with gearing reversal or clutch reversal, for example, allowing for extraction of material from the patient in a proportional amount as a function of the cannula 12 travel, thus harvesting a precise amount of material from the patient on a per “area” basis.

In an exemplary embodiment of the injection control device, the gearing arrangement of FIG. 3 is illustrated with the primary components of the positioning rack 24, engaging a positioning rack gear assembly 55. The positioning rack gear assembly 55 having an outer gear 54 and inner gear 56 and clutch (not seen) is coupled to a syringe rack gear 57 having an outer gear 58 and an inner gear 62 (not seen), which is engaged to the syringe rack 34. The positioning rack 24 is constrained and guided by positioning rack rollers/guides 25a, which are placed at strategic points along the travel area of the positioning rack 24, to guide and maintain smooth travel of the positioning rack 24 through the body 18. Similarly, syringe rack rollers/guides 34a are illustrated as guiding and/or constraining the syringe rack 34 within the body 18.

It should be appreciated that while FIG. 3 illustrates various rollers/guides 25a and 34a, disposed within and about the body 18, other forms or arrangements of rollers/guides that are known in the art or future-derived, may be used to achieve the desired effects, without departing from the spirit an scope of this disclosure. In fact, in some embodiments, the roller/guides 25a and 34a may be supplanted with full body guides along the body 18, such as a channel or sleeve. Since knowledge of such presently known rollers/guides and alternative arrangements are within the purview of one of ordinary skill in the art, they are not discussed herein.

In one mode of operation, the ring 22 is held stationary with respect to the skin. The body 18 of the injection control device is moved as the cannula 12 is withdrawn. In another mode of operation, it may be desirable to advance the entire injection control device as a unit as the cannula 12 is advanced into the tissue. Then the ring 22 is held stationary with respect to the skin as the body 18 of the injection control device with the syringe 14 and cannula 12 is withdrawn expelling the filler material. The ring 22 is then pushed back into the body 18 of the injection control device. The entire injection control device is then again advanced as a unit.

In another mode of operation, the reverse effect can be accomplished, wherein by advancing the cannula 12 into the skin, material can be “sucked” into the injection control device. Therefore, as will be apparent from the description provided herein, multiple modes of operations may be contemplated, accordingly, the injection control device may also operate as a suction (extraction) control device.

In view of various movements of the body 18 with respect to the ring/positioning guide 22, the positioning rack's teeth 24a will engage with the teeth 54a of the outer gear 54 of the positioning rack gear assembly 55 and cause rotation. The positioning rack gear assembly 55 may be configured with teeth ratios to act as a reduction gear in order to translate the linear displacement of the positioning rack 24 to a reduced linear displacement of the syringe rack 34. As the teeth 56a of the inner gear 56 of the positioning rack gear assembly 55 engage with the teeth 58a of the outer gear 58 of the syringe rack gear 57, the teeth 62a (not shown) of the inner gear 62 (not shown) will engage the teeth 34b of the syringe rack 34, causing a linear displacement of the syringe rack 34.

It is should be apparent from the above description concerning the operation of the ICD that the ring 22, when held against a patient's skin or surface, etc., operates to “fix” the position the end of the ICD and also, via its fixed connection to the positioning rack 24, forms a stationary reference point for the ICD's internal mechanics to react against. That is, the now “fixed” position of the positioning rack 24, being acted against by the ICD internal mechanics, facilities the conversion of the translation forces of the body 18 to motion of the syringe rack 34. It should also be apparent that since the syringe 14 is fixed to the body 18, as the body 18 is being translated the syringe's cannula 12 will also translate with the body 18. Consequently, as the cannula 12 is being translated in or out of the patient/subject, the exemplary ICD injects or extracts in synchronicity with the cannula's 12 movement. Thus, injection or extraction occurs while the cannula 12 is moving.

Regarding terminology, since the ring 22 can extend to, or in some embodiments, beyond the tip of the cannula 12, it can function as a positionable member to assist in aligning the cannula 12 to the patient or subject. Also, since the positioning rack 24 is fixed to the ring 22, the combination of the ring 22 and the positioning rack 24 operates as a reference member for the internal transmission (e.g., gearing assembly, etc.) to react against as the body 18 is translated when the ring 22/positioning rack 24 is stationary or fixed. Accordingly, it is understood the term “positioning guide” as used herein does not describe a member that solely operates for positioning an injection device, but a member that is extendable to a fixed location (positionable) on the patient/subject, and being fixed provides a reference point or fixture for the body and associated transmission to react. Therefore, while the term “positioning guide” is used throughout this disclosure, it is expressly understood that it describes a positionable transmission reference member.

In an exemplary embodiment of the injection control device, a ratio of approximately 5.2093:1 was used to effect the desired movement of the positioning rack 24 with respect to the syringe rack 34. That is, for every 5.2093 inches the injection control device is displaced or “withdrawn” from the tissue with the ring 22 held in place, the syringe rack 34 advances approximately 1 inch. Given a commercially available 1 cc syringe, the exemplary injection control device will inject approximately 0.00436 cubic inches of filler material for every one inch the cannula 12 is withdrawn from the tissue.

The gearing ratio described above may be adjusted according to methods and systems known in the art of gearing. Therefore, the gearing ratio may be adjusted by simply replacing the appropriate gears and racks to achieve a desired injection rate. In such embodiments, a “dialing” in of a different gear ratio may be contemplated, according to gearing systems known in the art. Alternatively, to achieve a different or variable injection rate, varying syringes with different bore diameters may be used, to increase or decrease the rate of material injected. If the outside diameter of the syringe is held constant while the internal diameter is varied, this will allow the effective gear ratio or “injection rate” to be easily varied according to the application. This can prove to be a very economical way of “changing gears” without changing the actual gearing of the injection control device or switching to a similar injection control device with a different gear ratio.

As is made apparent from the above description, one mode operation of the exemplary injection control device may entail the practitioner positioning the injection control device with the ring 22 (operating as a positioning guide) against the skin or a pre-determined distance from the skin of a patient. With the ring 22 (positioning guide) held in a stationary position, the body 18 of the injection control device can be advanced into the tissue surrounding the skin and then withdrawn, with the ring 22 (positioning guide) held in place. Consequently, the advancing motion of the cannula 12 will create a tract in the tissue, while the withdrawing motion of the cannula 12 (the body 18 of the injection control device) will deposit the filler material in the void created in the tract as the cannula 12 is withdrawn.

In order for the ring 22 to be fixed at a desired position in proximity to the skin or surface of the tissue, the ring 22 should be allowed to be manipulated in a “forward” or skin-side direction without causing the syringe rack 34 to move. This freedom is achieved by a clutching mechanism that is discussed in further detail below.

It should be appreciated that, in some embodiments, it may be desirable to have the ring 22 (positioning guide) flush to the skin, thus providing the stable reference of the skin surface or body surface for the practitioner to exert a “push” against while he is “pulling” the injection control device. Of course, it should be apparent that depending on the preferences and skills of the practitioner, the ring 22 may not placed against the skin or surface but at a preferred distance. For example, a practitioner may place his thumb into the ring 22 and use the span of his hand with his fingers or palm against the skin, resulting in the ring 22 being positioned a pre-determined distance from the surface of the tissue. Thus, it should be apparent that variations of the placement of the ring 22 as well as its shape may be practiced without departing from the spirit and scope of this disclosure.

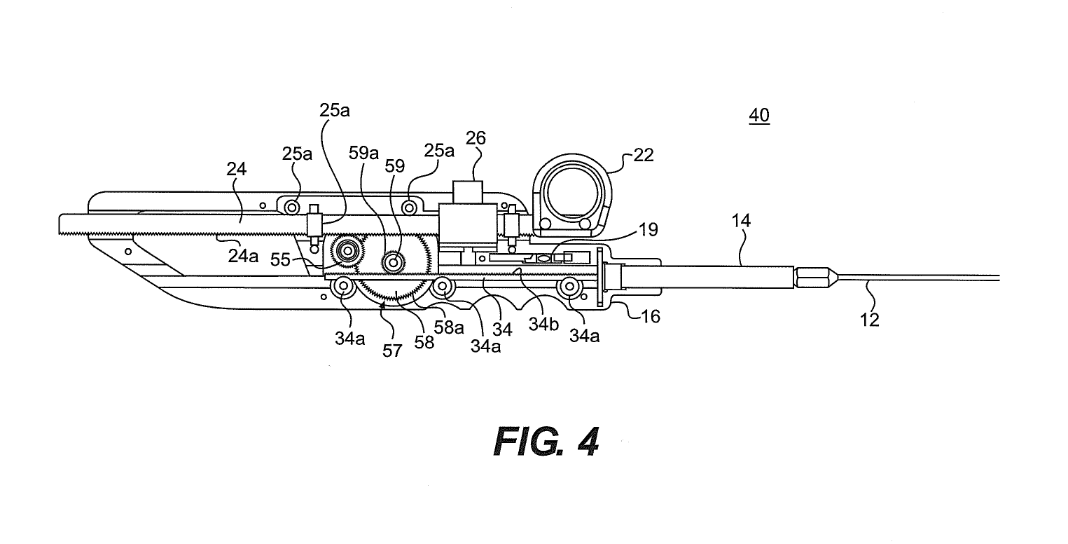

FIG. 4 is a close-up illustration 40 of the reversed side of the interior of the exemplary injection control device. FIG. 4 illustrates the teeth 59a of the syringe rack gear 57 engaging the teeth 34b of the syringe rack 34.

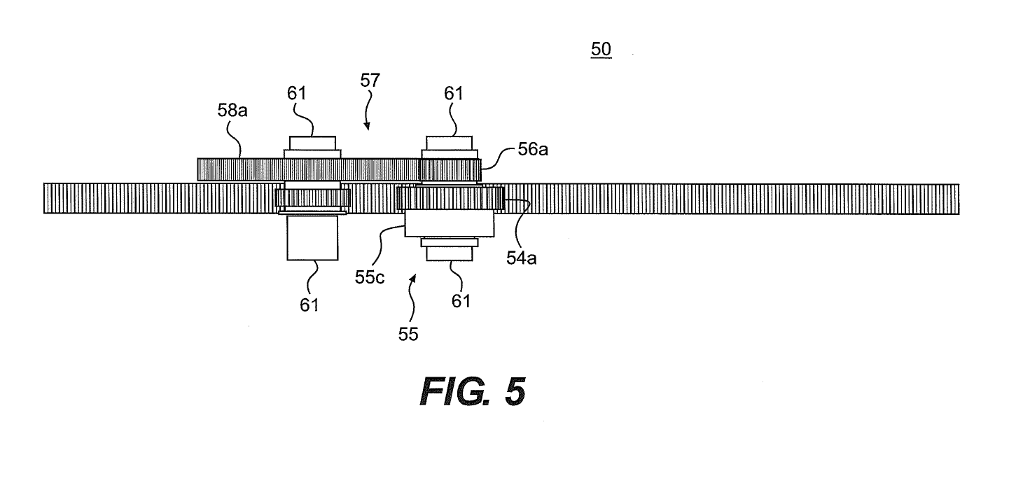

FIG. 5 is a bottom-side illustration 50 of the gear contacts of the exemplary injection control device with the syringe rack 34 removed from view. The positioning rack gear assembly 55 is shown with a clutch 55c which acts as an intermediary between the outer gear 54 and the inner gear 56 of the positioning rack gear assembly 55. The clutch 55c functions to provide a mechanism to enable “free” movement of the positioning rack 24 without causing the inner gear 56 of the positioning rack gear assembly 55 to move. Thus, the positioning rack gear may be moved in a preferred direction without causing the syringe rack gear 57 to turn. In principle, the clutch 55c allows advancement of the syringe plunger into the syringe cylinder but not its withdrawal. Therefore, the clutch 55c allows the injection control device to be advanced relative to the ring 22 without causing the plunger to move relative to the syringe cylinder. In other embodiments, the clutch 55c can be configured to allow retraction of the syringe plunger from the syringe cylinder without engaging or causing the positioning rack 24 to move.

As shown in FIG. 1, the brake 26 may be used to stop or engage the motion of the positioning rack 24. Therefore, by engaging the brake 26, the ring 22 may be secured while the cannula 12 is positioned in the tissue. It should be noted that the brake 26, in some embodiments may not be necessary, as operation of the injection control device can conceivably be executed without use of the brake 26.

In particular, the use of a clutch 55c or one-direction-engagement mechanism enables the practitioner to adjust the position or extension of the positioning rack 24 from the body 18, with the ring 22 at a desired distance from the patient's tissue, without causing the syringe rack 34 to move in a reverse orientation. The clutch 55c can be engaged in such a manner to cause the gear train to rotate and advance the syringe rack 34 (or plunger) into the syringe, as the body 18 of the injection control device is moved away from the ring 22. The clutch 55c allows the body 18 of the injection control device to move towards the ring 22 without the syringe rack 34 moving with respect to the syringe. Also, the clutch 55c can be configured to prevent the gear train from moving the syringe rack 34 with respect to the syringe as the body 18 is advanced with respect to the ring 22.

In some embodiments, the clutch 55c may be supplanted with an arrangement wherein the teeth 54a of the outer gear 54 are displaced from the teeth 24a of the positioning rack 24, by some switch or motion (not shown) that is coupled to the positioning rack gear assembly 55. Thus, by removing contact of the teeth 54a of the outer gear 54 from the teeth 24a of the positioning rack 24, the positioning rack 24 may be moved without causing the syringe rack 34 to move.

It should be appreciated that one of ordinary skill in the art of gearing may devise an alternative scheme for providing “free” movement of the positioning rack 24 in a preferred direction, or even in both directions. The above clutching mechanism 55c is provided as one simple scheme for achieving the desired results wherein more complicated or different schemes may be contemplated. Therefore, other schemes or systems for providing controlled motion or contactless motion may be used, whether using gears, clutches, slips, discs, springs, etc., without departing from the spirit and scope of this disclosure.

FIG. 5 also illustrates the use of gear axle caps 61 for the positioning rack gear assembly 55 and the syringe rack gear 57. It should be appreciated that in some embodiments, the gear axle caps 61 may not be necessary, as axle securing methods not consisting of caps 61 may be used, such as those that are common in the industry. Additionally, the illustrated spacing between the gears and rack(s) shown may be adjusted according to design preference.

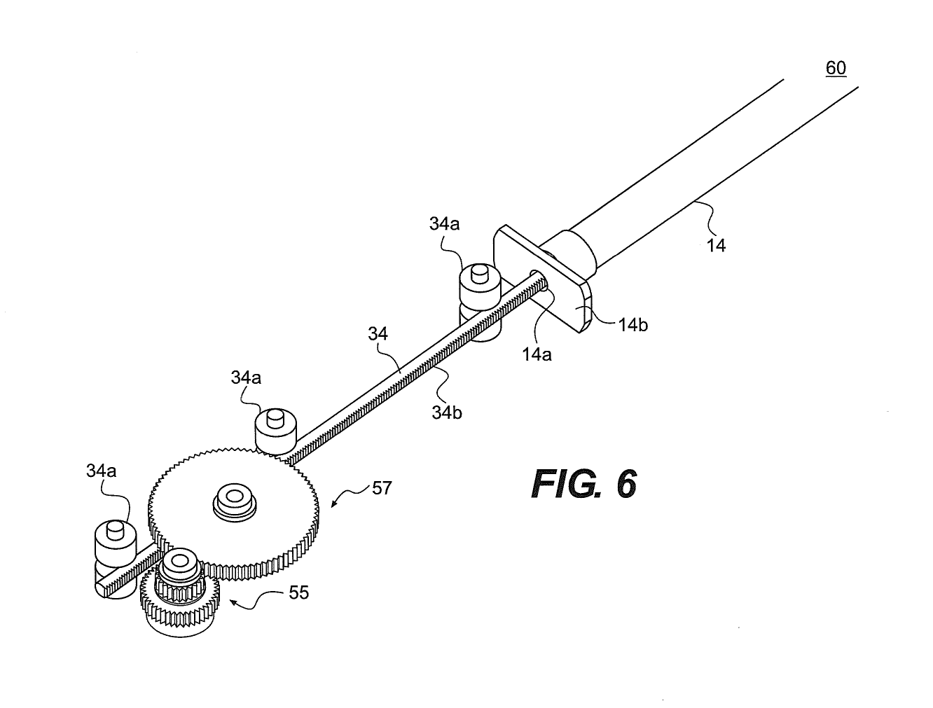

FIG. 6 is a perspective view illustration 60 of the syringe rack arrangement. Specifically, the syringe rack 34 is illustrated with a smooth ridge 34b that fits within a channel within the roller/guides 34a. By use of the smooth ridge 34b within the channel, lateral movement of the syringe rack 34 can be minimized. Of course, in some embodiments, the roller/guides 34a may be replaced with bearings, if desired. Or, the ridge 34b may be replaced with a channel “under” the syringe rack 34, wherein bearings or roller/guides may be disposed. In some embodiments, the syringe rack 34 may have a different shape, according to design preference. Therefore, round, square, rectangular or other shapes may be used. Also, a non-bearing configuration, using for example, the interior of the body 18 as a constraining and guiding entity may be used. Therefore, alternative arrangements for guiding the syringe rack 34 may be used without departing from the spirit and scope of this disclosure.

The syringe rack 34 is also shown in FIG. 6 as having its “front” plunger end inside an opening 14a of the syringe 14. In some embodiments the syringe rack 34 may be configured to drive another mechanism that acts as a plunger for the opening 14a of the syringe 14. Thus, some form of pivoting may be designed to cause the syringe rack 34 to move “outside” the opening 14a, while still achieved the desired effect of moving a plunger into or out of the syringe 14. In some embodiments, the syringe rack 34 may be an integral part of the syringe 14. That is, the syringe rack 34 may constitute the actual plunger mechanism in the syringe, or a controlling member. Thus, a syringe 14 may be configured with a syringe rack 34 pre-configured for use with the injection control device. Alternatively, the syringe rack 34 may be configured with a geometry that is suitable for use with disposable syringes. Therefore, the injection control device may use disposable syringes or may use syringes having a plunger with a syringe rack 34 attached. Moreover, the ICD itself may be designed to be disposable after a single use, or single procedure.

It should be noted that in FIG. 6, the anterior end of the syringe 14 is shown having flanges 14c. The typical syringe 14 is understood to have such flanges 14c, and therefore, in various embodiments the injection control device exploits the presence of the flanges 14c by accommodating them in bulged areas of the syringe supporting section 16. In some embodiments, the syringes 14 may not have such flanges 14c, therefore an appropriate securing mechanism may be devised, such as a clamp or well, for example, for securing the syringe 14 to various embodiments of the injection control device. In such embodiments, the flanges 14c may be of a reduced size and therefore, the upper body 18a and lower body 18b portions surrounding the flanges 14c may be altered in a manner suitable for achieving the desired effect, without departing from the spirit and scope of the disclosure

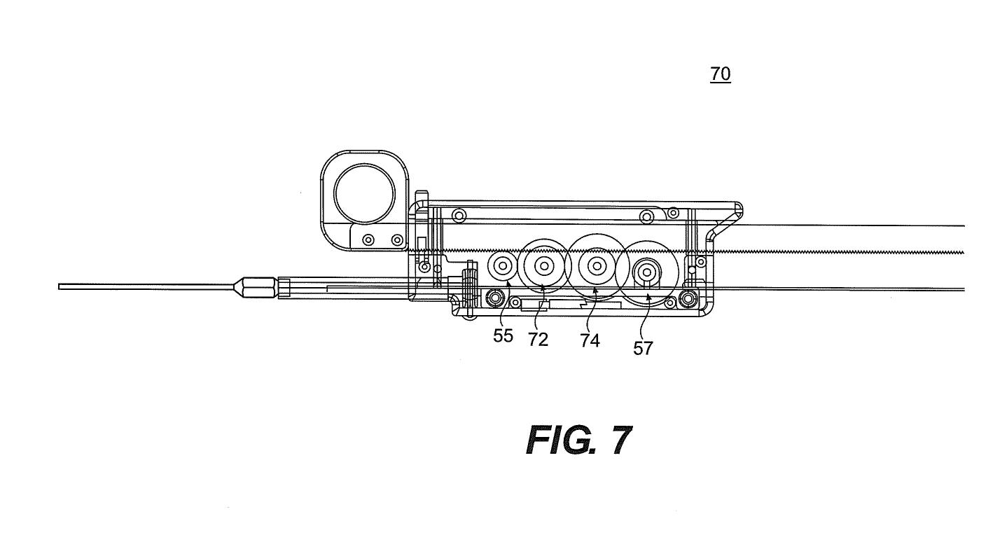

FIG. 7 is an illustration 70 of the outline of an embodiment of an injection control device with multiple gears. Specifically, this embodiment of the injection control device is illustrated with four gears, chaining action from the first positioning rack gear assembly 55 to a series of “reduction” gears 72 and 74, to the syringe rack gear 34. By use of multiple gears 72 and 74, varying amounts of ratios can be achieved. Of course, while FIG. 7 illustrates a total of four gears in the gear train, more or less gears may be used according to design preference.

By use of the injection control device several advantages can be obtained:
The injection of the filler material is substantially proportional to the length of the injection tract and uniform along the course of the injection tract;
An “automatic” controlled injection system can be used for fat grafting or injection of other filler materials;
Intracutaneous, subcutaneous and intramuscular injections of filler materials can be precisely controlled;
A fixed amount of fat or other filler material can be injected per unit distance traveled by the tip of the cannula;
The injection ratio (amount of material injected over a given distance of cannula withdrawal) can be varied by simply using varying bore diameter syringes;
The use of syringes (disposable); and
The use of syringes incorporating a rack in the plunger.

It should be appreciated that based on an understanding of various embodiments of the injection control device disclosed herein, several modifications may be contemplated without departing from the spirit and scope of this disclosure. As some cannulas may be of different diameters and openings, a volume approach may be achieved by adjusting the gearing, for example.

As another modification, the clutch 55c may be configured to operate in a “reverse” manner than described. That is, rather than having an embodiment of the injection control device inject filler material, the injection control device may be configured to “suck” filler material. Thus, in some applications, harvesting of fat or filler material may be accomplished by altering the clutching or gearing of the injection control device.

Along the lines of the above modification, it is possible to design a gearing system that injects filler material as the cannula is advanced, rather than withdrawn. Additionally, an embodiment of the injection control device may be configured with opposing gear trains that would enable the injection of filler material as the cannula is advanced as well as when the cannula is withdrawn. Similarly, an embodiment of the injection control device may operate in a manner to enable the withdrawal or sucking of filler material as the cannula is advanced as well as when the cannula is withdrawn.

Several other variations of embodiments of the injection control device described above are detailed below.

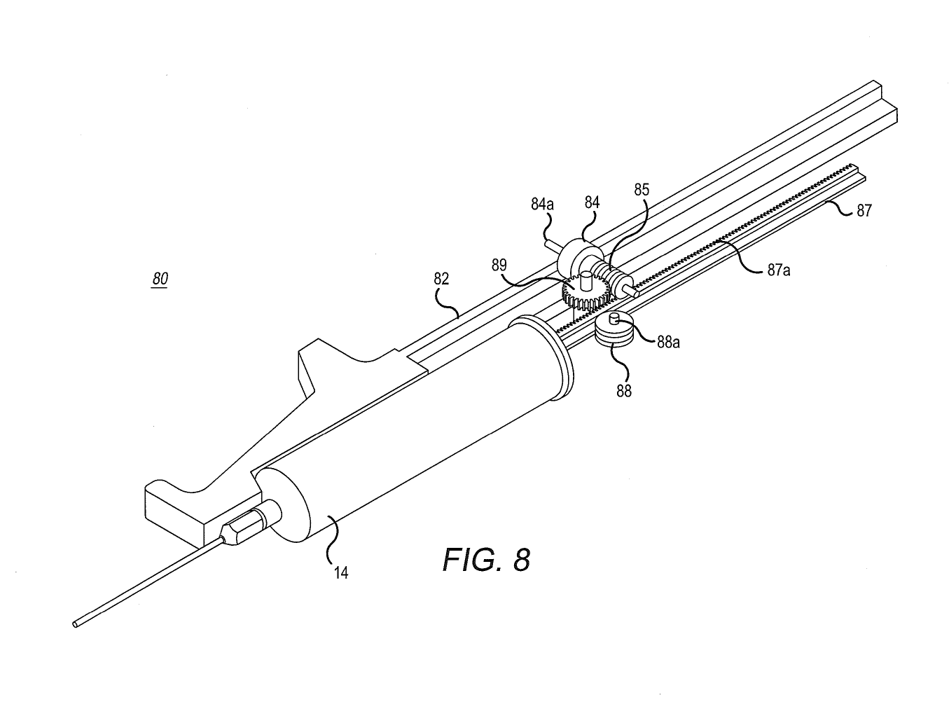

FIG. 8 is an illustration 80 of a perspective bodiless view of an embodiment of an “rackless” injection control device according to a second configuration. The body is removed from view so the internal mechanisms can be seen, recognizing that syringe 14 is fixed to the removed body. The general principles of operation are similar to the previous embodiment/configuration, but with a rackless positioning guide 82. In this FIG. the syringe-side of rackless positioning guide 82 is also shown in close proximity to the body of syringe 14, shielding one side of the syringe 14. The rackless positioning guide 82 is achieved by use of a spooling mechanism 84 coupled to rackless positioning guide 82 with concentric worm gear 85 that engages main gear 89. Spooling mechanism 84 contains a clutch or locking/unlocking mechanism (not shown) controlling worm gear's 85 ability to rotate with spooling mechanism 84. Roller bearings used to support the positioning guide in the previous embodiment can be replaced by sleeve bearings (not shown) in the removed body. Axle 84a of spooling mechanism 84, axle 89a of main gear 89, axle 88a of bearing 88, and syringe 14 are secured to removed body, so that these elements travel with the body as the body is translated.

Thus, with the clutch is engaged, worm gear 85 rotates with spooling mechanism 84 as the removed body is translated with respect to rackless positioning guide 82. Spooling mechanism 84 will unwind, turning worm gear 85 which turns main gear 89, which engages teeth 87a of plunger rack 87 to drive or retract the stopper (not shown) in the syringe 14. Plunger rack 87 may be supported by a single bearing 88. When the clutch is not engaged, spooling mechanism 84 may rotate without causing rotation of worm gear 85. It is noted it is possible that the resistance of the stopper in syringe 14 will operate to obviate the need for a second clutch to prevent movement of the plunger rack 87 during preliminary setup of the ICD.

Spooling mechanism 84 may be a drum with a coil or a constant force spring, for example. Coil portion (end of) the constant force spring can be can be attached to the forward or aft section of rackless positioning guide 82, depending on the mode of operation. The constant force spring provides tension on the coil to allow it to wind properly. Further, if enough tension is provided, the winding force may be sufficient to assist in driving (or withdrawing, depending on mode of operation) the plunger rack 87 back to its starting position. For injection/extraction materials that are particularly viscous or thick, the implementation of an assistive device (such as the constant force spring) can be beneficial. It is envisioned, in some embodiments the positioning guide 82 may start in the extended position and via insertion of the cannula 12 into the tissue, the insertion force operates to push the positioning guide 82 into the retracted position and loads the constant force spring, which in turn provides the motive force to drive the gear train, as the positioning guide 82 is allowed to push the body of the ICD away from the subject being injected. Accordingly, a constant force spring or spring motor could be loaded by a winding mechanism to store energy that could be used to assist in the injection.

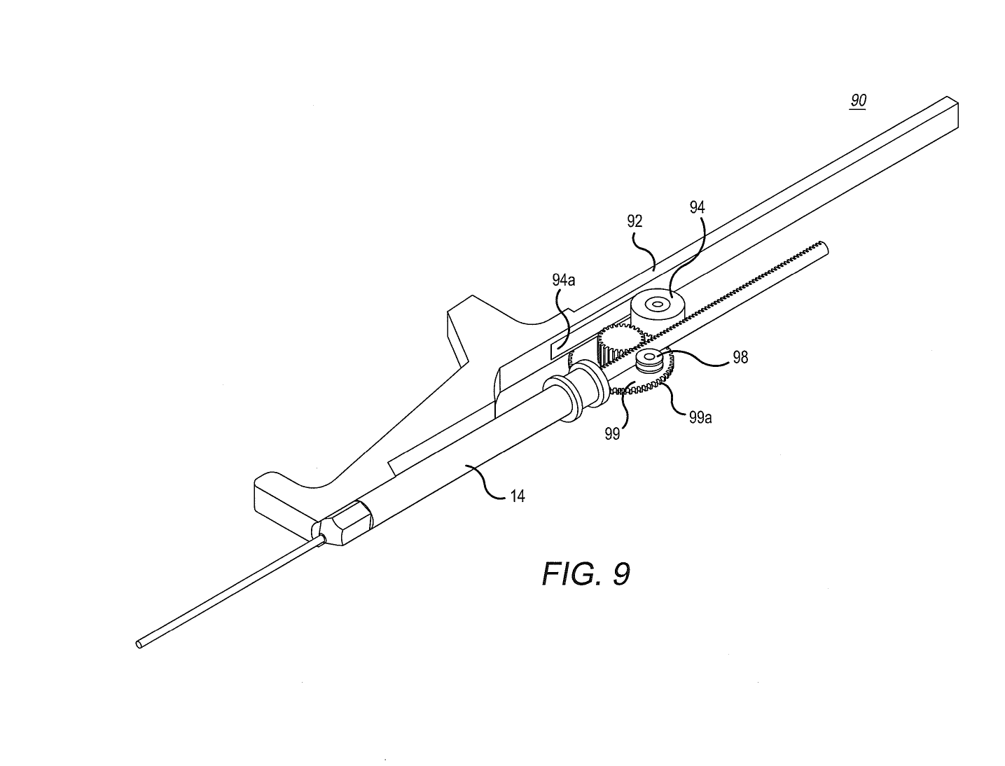

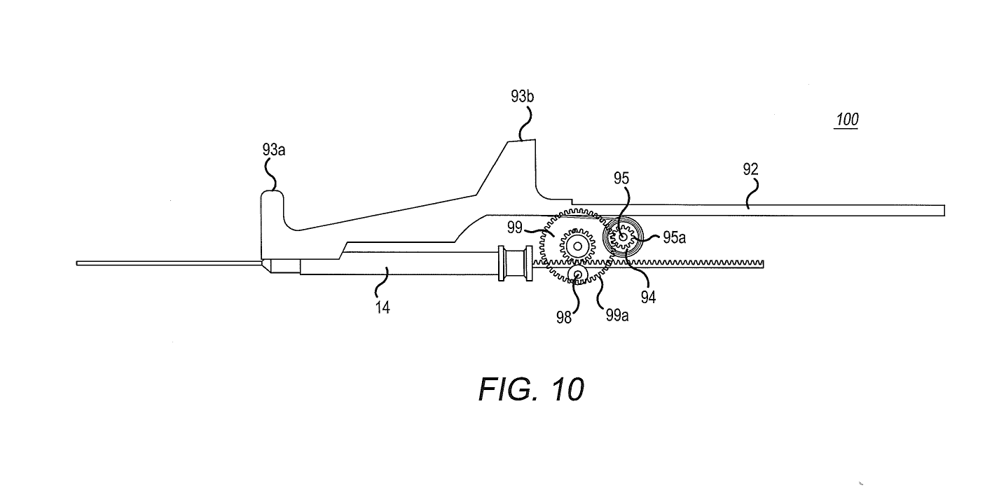

FIGS. 9-10 are illustrations of another embodiment of a bodiless “rackless” exemplary injection control device according to a third configuration. FIG. 9 is an illustration 90 of a perspective view and FIG. 10 is a side cut-way view 100. These FIGS. show a variation of the embodiment/configuration shown in FIG. 8. As seen in FIG. 9-10, instead of using a worm gear, spooling mechanism 94 (or constant force spring, for example) is joined with a coaxial gear 95 having teeth 95a that directly contact main gear 99 via main gear's outer teeth 99a, to drive main gear 99. In this embodiment, constant force spring is used as the spooling mechanism 94 and is aligned in the same orientation as the main gear 99 to obviate the need for a worm gear as well as reduce the overall “thickness” of the gearing assembly. Coil of the constant force spring is attached 94a to a side of rackless positioning guide 92. The subsequent mechanics of motion for operation of the ICD are similar to those described in FIG. 8.

It is worthy to note in passing that similar to FIG. 8, the forward portion of the positioning guide 92 is configured without a “thumb” or “finger” hole, but is configured with two open extensions 93a and 93b. The openness of these extensions allows them to be pressed against using a palm or fingers. For example, extension 93a can be “pushed” forward using a palm resting against the extension, while extension 93b can also be “pushed” forward via a palm or fingers. Conversely, extension 93b can be large enough to be gripped with a hand to be “pulled” back (i.e., retracted), if the mode of operation requires such a motion. A certain increased ease of handling is obtained by having open extensions versus a closed extension (thumb or finger hold such as seen in the first embodiment). While palm, fingers, hands are described as pulling or gripping to cause the desired motion, it is understood that in various embodiments of the injection control device may be actuated using other means and ergonomics including but not limited to a trigger squeeze mechanism or a hand squeeze mechanism.

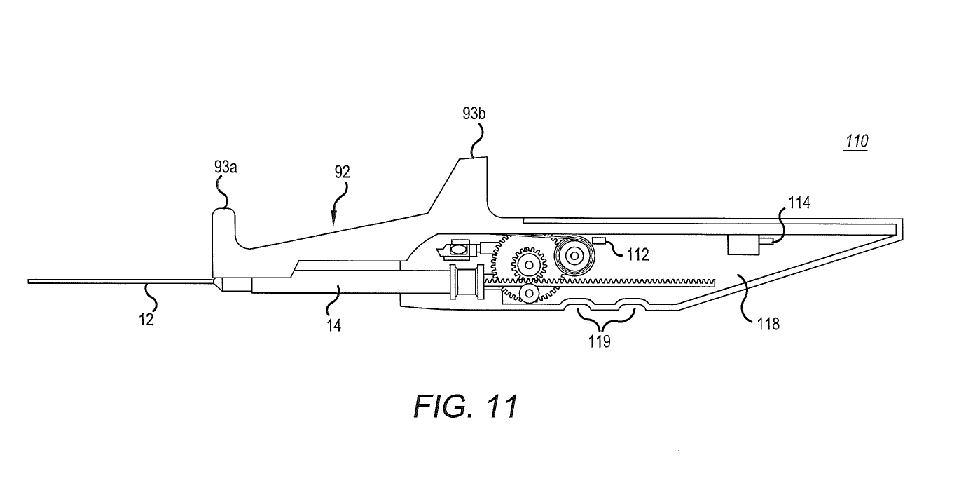

FIG. 11 is an illustration 110 of an internal side cut-away view of embodiments of the injection control device of FIGS. 9-10 but with body 118 and one or more adjustable stops 112, 114. The adjustable stops 112, 114 operate to limit the range of motion for the body 118 and/or the positioning guide 92 as it slides through body 118. This “control” effectively limits the distance the cannula 12 will travel with respect to the positioning guide 92. Use of the adjustable stops 112, 114 will precisely control the distance over which the deposition/extraction of material occurs.

The adjustable stops 112, 114 can be a pin that is inserted in multiple accommodating “holes” (not shown) along the body/positioning guide or a sliding lock as seen in disposable box cutters. Of course, other forms or mechanisms for locking or restricting the range of motion may be used and are understood to be within the purview of one of ordinary skill.

Also evident in this FIG. is that, for this example, the front 93a of positioning guide 92 has been designed with a substantially flat surface, thus conceivably acting as a “depth” gauge, preventing insertion of the cannula 12 past a certain point on the cannula 12. In regard to gauges, this or other embodiments of the ICDs may have a gauge (not shown) external or visible on the body 118, to allow the practitioner to view the amount of material in the syringe 14. In some embodiments, the body 118 may have an opening (not shown) that allows viewing of the syringe 14. To this end, body 118 does not completely encase syringe 14, as in the first embodiment. Rather the bulk of the syringe 14 is exposed, which allows for the practitioner the ability to visually inspect the syringe's contents, before and after administration. As in previous embodiments body 118 is configured with optional finger rests 119.

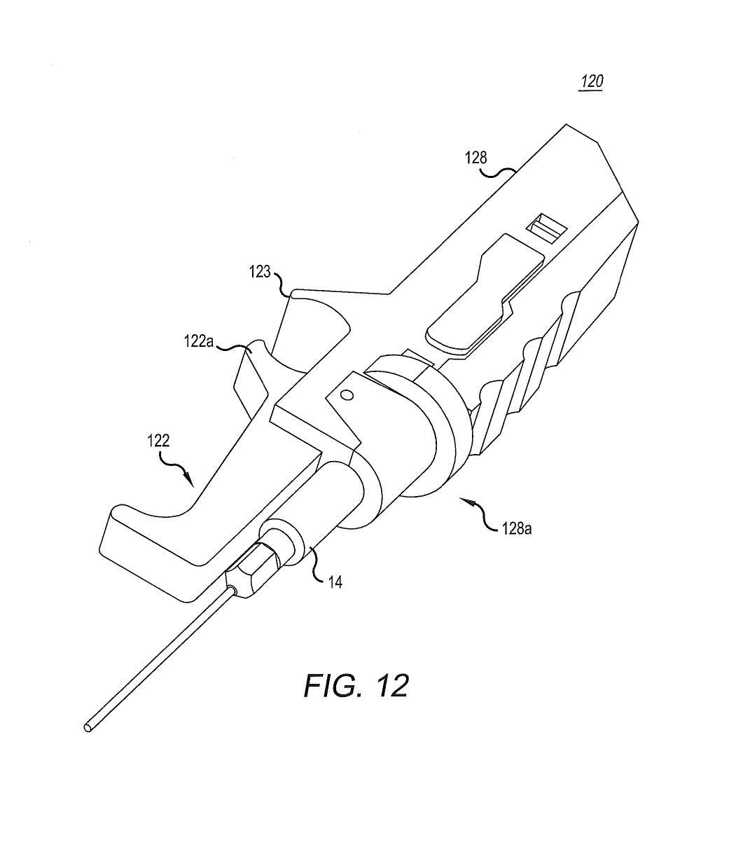

FIG. 12 is an illustration 120 of a perspective view of an embodiment of an injection control device according to a fourth configuration. The body 128 is shown with a thumb rest 123 to assist in gripping the ICD as the cannula 12 is advanced into the tissue. Thumb rest 123 can also operate as safety ridge to reduce accidental pressure on the exposed rear portion 122a of positioning guide 122. Thumb rest/safety ridge 123, depending on design preference, can also operate as a fixed stop for positioning guide 122, restricting the amount of retraction available to positioning guide 122. Body 128 can also be accommodated with a separable lower portion 128a that allows for access to the interior of body 128, so as to insert the syringe 14 into the ICD. In some embodiments, the body 128 can be configured into a clamshell design where the bottom half rotates on an axis located in the end of the device away from the cannula 12.

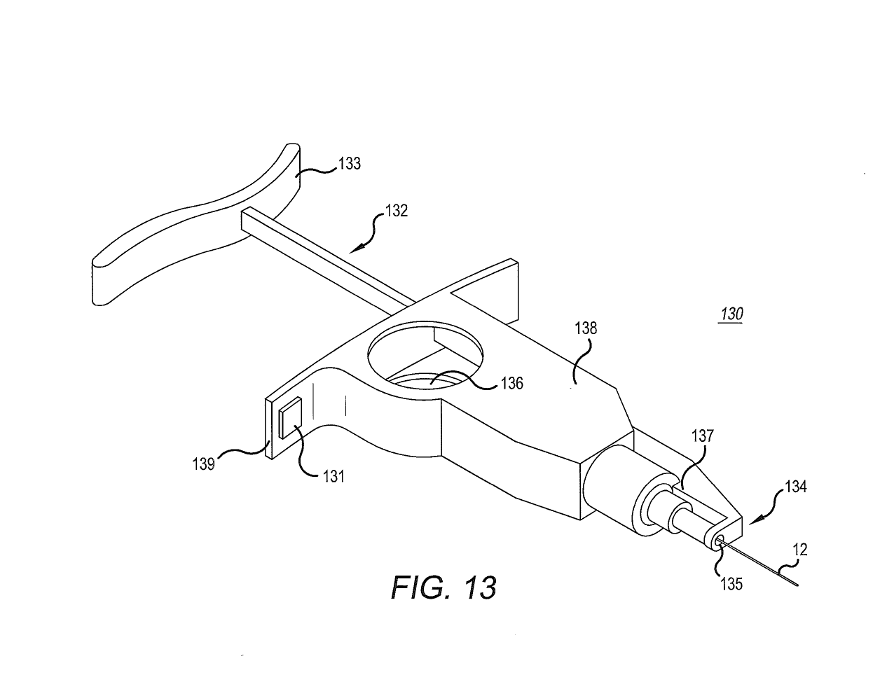

FIG. 13 is an illustration 130 of a perspective view of an embodiment of the injection control device according to a fifth configuration. In this embodiment/configuration, a design is described that allows the user to operate this embodiment of the ICD with one hand, in a manner similar to operating a typical syringe. In this embodiment, distal portion of positioning guide 132 is configured with a butt plate 133 and proximal portion of positioning guide 132 is configured with a contact plate 134 with an aperture 135 to accommodate the passage of the cannula 12. The positioning guide 132 is contiguous, spanning the butt plate 133 to the contact plate 134. The aperture 135 may be interior to contact plate 134 or on an edge of contact plate 134, forming an opening on a side of contact plate 134.

The butt plate 133 can be large (as shown in this FIG.) or small, depending on design preference. Further, butt plate 133 can be of any desired shape that allows a user to facilitate contact with the user's thumb or palm, when operating the ICD (for example, similar to how a syringe is operated). The positioning guide 132 is shown as traveling through a portion of body 138 (so as to engage interior “gearing”), the body 138 being configured with finger recess 136 and finger and/or grip rest(s) 139. Depending on how large finger and/or grip rest(s) 139 are made, body 138 can be configured with a single grip rest 139 that is pistol grip-shaped. The design of this embodiment facilitates the easy manipulation of the ICD with a single hand, potentially freeing the practitioner's other hand for other treatment-related actions.

In order to prevent movement of the syringe or mechanics of the ICD during initial set up (e.g., injection into subject), a trigger lock 131 may be utilized, for example shown here as being optionally situated on finger and/or grip rest(s) 139. In one state, trigger lock 131 can lock the syringe within body 138 as cannula 12 is advanced into the tissue, for example, in an un-pressed state. Pressing finger and/or grip rest(s) 139 while pressing trigger lock 131, “unlocks” the ICD's mechanics to allow body 138 to be compressed towards butt plate 133. Locking/unlocking the ICD's mechanics can be via control of a clutch, or an obstruction to prevent any gearing from rotating, or the positioning guide 132 from movement. Numerous means of “locking/unlocking” are known to one of ordinary skill in the art, therefore, modifications may be made without departing from the spirit and scope of this disclosure.

Alternatively, in an extraction mode of operation, trigger lock 131 may operate in a reverse manner. Depending on design preference, detent 137 in positioning guide 132 can operate as a stop for body 138, limiting its forward motion. Similarly, butt plate 133 can operate as a stop, limiting body's 138 rearward motion. Detent 137 can be made to be adjustable by any one or more means known to one of ordinary skill. As one non-limiting example, a rotating nut, as seen in adjustable crescent wrenches, can be used to adjust the position of detent 137. Alternatively, rear surface of contact plate 134 may also operate as a stop against forward movement of body 138, if so desired. Therefore, multiple forms of “stops” may be developed either on body 138 or positioning guide 132. Accordingly, it is understood that modifications can be made to the form and type of stops used without departing from the spirit and scope of this disclosure. Similarly, while FIG. 13 illustrates a particular shape of body 138, other suitable shapes may be contemplated or used without departing from the spirit and scope of this disclosure.

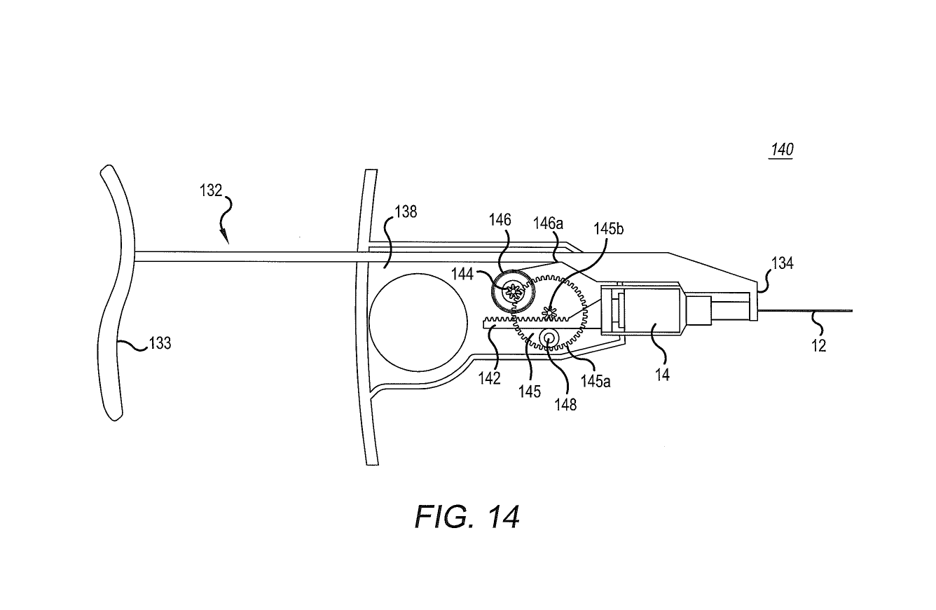

FIG. 14 is an illustration 140 of a side cut-away view of the embodiment of the injection control device of FIG. 13. As multiple implementations of trigger locks (described in FIG. 13) are possible, they are not illustrated in FIG. 14, but are understood to incorporable, as according to design preference. Positioning guide 132 is shown with butt plate 133 being bridged to contact plate 134 via a single contiguous connection. Positioning guide 132 traverses the entire length of body 138 to extend to the front of syringe 14, shown here as terminating at the base of cannula 12. In some embodiments, it may be desirable to have contact plate 134 “adjusted” to terminate at variable locations on cannula 12, depending on insertion depth parameters per application. Accordingly, variability of contact plate 134 positioning may be implemented, according to design preference.

Syringe 14 is fixed to the forward (or proximal) portion of body 138 with plunger rack 142 operating as the plunger for the syringe 14. Plunger rack 142 can be “supported” or guided by bearing 148 in body 138. Gear 144 that is secured to body 138 engages main gear's 145 outer teeth 145a. Gear 144 is rotated via a fixed connection 146a to positioning guide 132. In this example, connection 146a is facilitated by a coaxial constant force spring 146. Not shown, optional clutch may be configured with gear 144 (with or without constant force spring) to allow motion in a preferred orientation (e.g., clockwise or counterclockwise).

As body 138 is translated with respect to the positioning guide 132, gear 144 will cause main gear 145 to turn, which, via main gear's 145 inner teeth 145b, contacts plunger rack 142 to cause it to move. FIG. 14 shows an embodiment that is configured for “injecting.” This embodiment an easily be converted to “extraction” by either having a clutch reversed and/or incorporating a secondary reversing gear between gear 144 and main gear 145, for example. Further connection 146a may be reversed in location and/or constant force spring 146 oriented to assist in extraction. Accordingly, modifications may be made to this embodiment to reverse its mode of operation without departing from the spirit and scope of this disclosure.

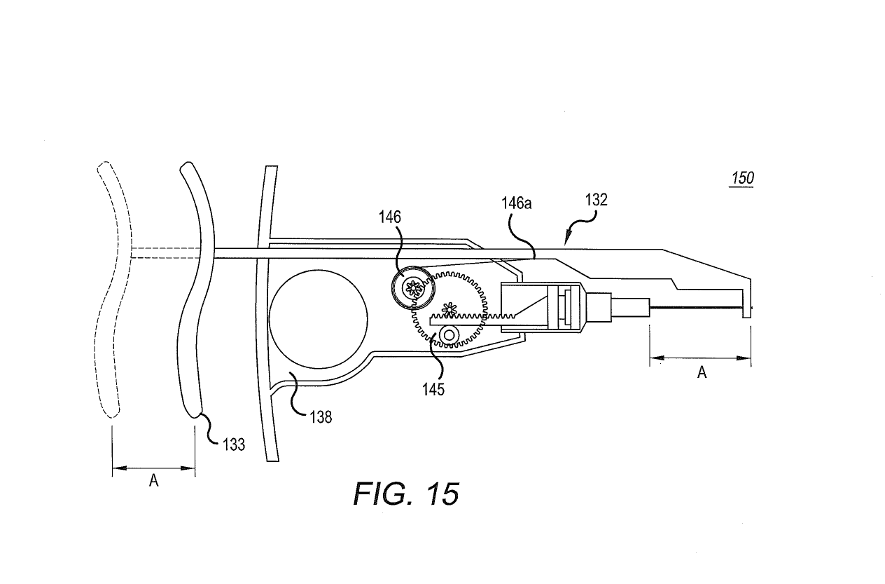

FIG. 15 is an illustration 150 of a side cut-away view of the embodiment of the injection control device of FIG. 14 in an extended position with body 138 retracted and with spooling constant force spring 146 “fully” unwound. The constant force spring 146 can be implemented, in some embodiments, to assist in reducing the amount of force needed to operate the ICD. In other embodiments, it can be used to provide a restorative force to bring the ICD (positioning guide 132) back to its original position, as discussed in the second embodiment. For example, depending on the amount of force provided by the constant force spring 146, the ICD can be designed such that as the user releases his thumb from the butt plate 133, the constant force spring 146, being unfurled and having tension between body 138 and positioning guide 132, automatically retracts the positioning guide 132 to return the ICD to its initial state (seen in FIG. 14). As the mechanics of this embodiment were described in FIG. 14, no further elaboration is needed.

It is noted, however, the design of the fifth embodiment is such that the amount of displacement of cannula 12 directly corresponds to the amount of displacement of the positioning guide 132 with respect to body 138. That is, distance “A” between open/closed positions of positioning guide 132 will be the maximum distance “A” that the cannula 12 can travel.

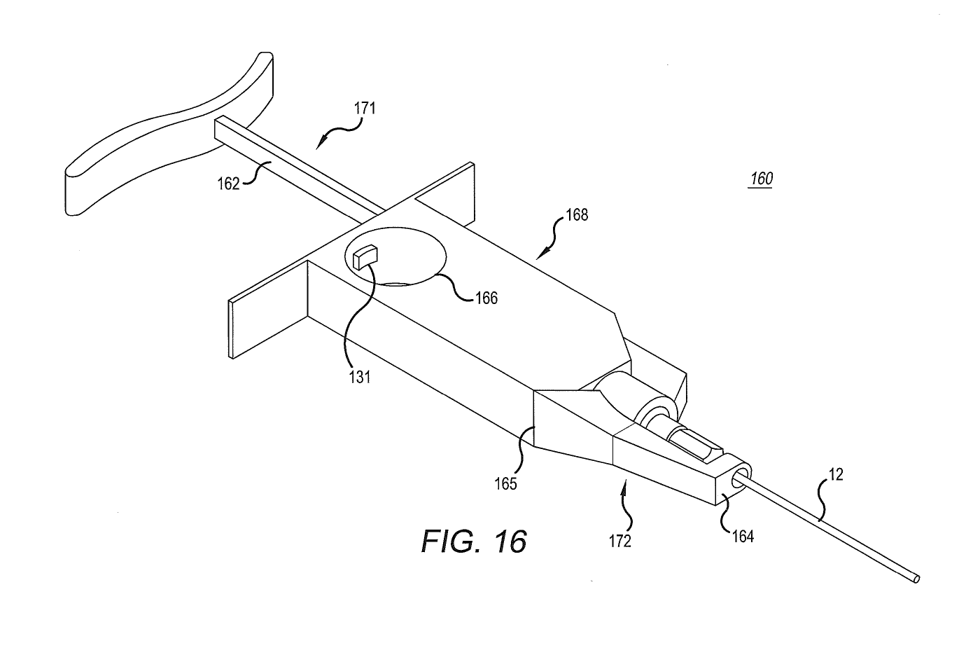

FIG. 16 is an illustration 160 of a perspective view of an embodiment of an injection control device according to a sixth configuration. FIG. 16's embodiment/configuration extends on the principles introduced in the fifth embodiment/configuration but provides an ability to increase (or decrease) cannula's 12 travel distance, by making positioning guide 162 non-contiguous. Specifically, positioning guide 162 is split into two sections 171 and 172 with ratioed “gearing” between the sections (as further discussed below), distance traveled by section 171 will differ from distance traveled by section 172. Body 168 accommodates “lower” section 172 with contact plate 164 via opening 165. Optional trigger lock 131 is illustrated here as being inside recess 166. Further details of this embodiment are presented below.

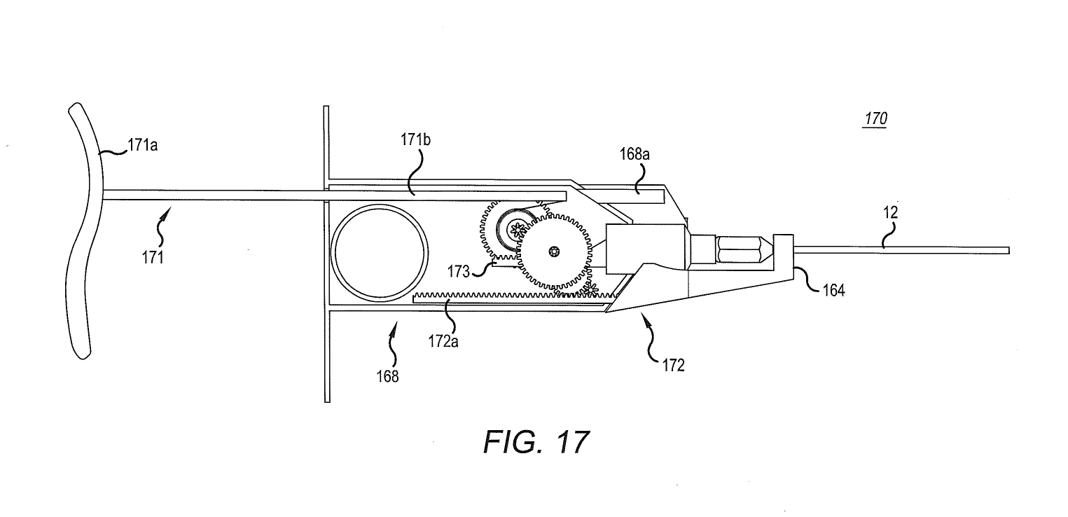

FIG. 17 is an illustration 170 of a cut-away view of the embodiment of the injection control device of FIG. 16 showing positioning guide 162 having an upper section 171, comprising butt plate 171a connecting a rackless pushrod 171b extending into body 168, having void 168a for accommodating end of rackless pushrod 171b; and lower section 172, comprising rack 172a extending out of body 168 connecting contact plate 164. “Upper” positioning guide section 171, being rackless operates, with respect to the plunger 173 in the same manner as described in the applicable above embodiments—for example, with spooling mechanism with main gear interaction. However, the upper positioning guide section 171 operates with the lower positioning guide section 172 via an indirect coupling mechanism (e.g., an separate gear train) that is rigidly fixed to the spooling mechanism, which turns in both directions, causing the lower positioning guide section 172 to extend and retract as the butt plate 171a and attached pushrod 171b are depressed and released. Because the indirect coupling mechanism, illustrated here as a gear train, is used between the upper positioning guide section 171 and lower positioning guide section 172, a gear ratio can be utilized to increase (or even decrease) the linear displacement of the lower positioning guide section 172 as compared to the upper positioning guide section 171. Additional details to the mechanics are described below.

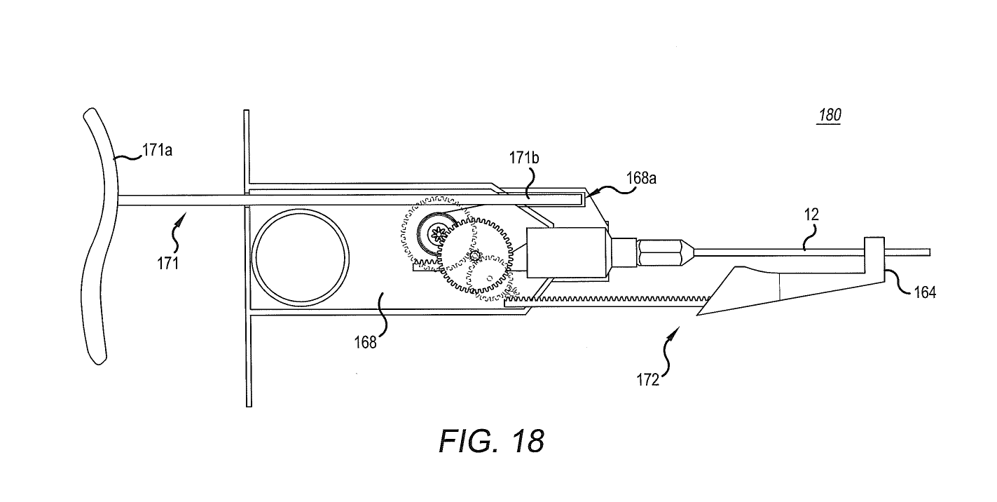

FIG. 18 is an illustration 180 of a cut-away view of the embodiment of the injection control device of FIG. 17 in an extended position, showing pushrod 171b of upper positioning guide section 171 depressed into void 168a, and lower positioning guide section 172 extended from body 168. Aspects of the indirect coupling mechanism that provides a proportional ratio is described below.

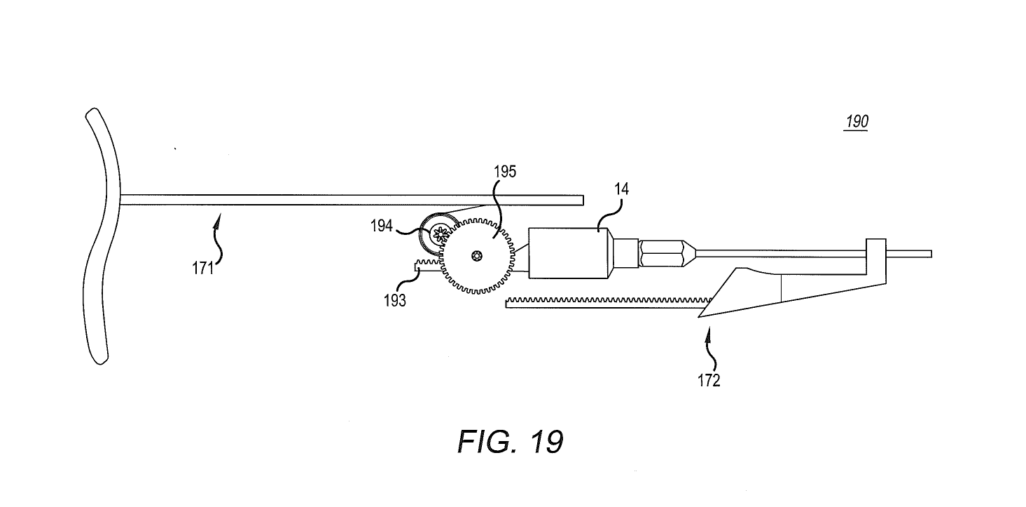

FIG. 19 is an illustration 190 of a cut-away view of the embodiment of the injection control device of FIG. 18, with the body removed showing the plunger activating gear train. “Upper” positioning guide section 171 controls the “amount” of material injected/extracted in syringe 14 via the spooling mechanism gear's 194 contact with main gear 195, which is in contact with the plunger rack 193. Operation of the plunger activating gear train is analogous to that of one or more of the previous embodiments, and is understood to be self-evident.

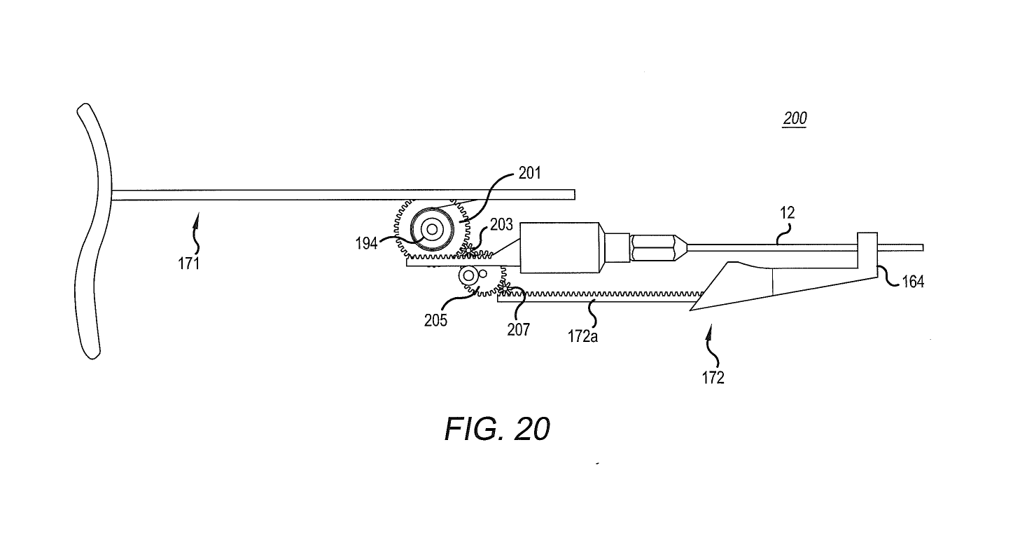

FIG. 20 is an illustration 200 of a cut-away view of the embodiment of the injection control device of FIG. 18, with the body removed showing the positioning guide gear train, comprising primary gear 201 coupled to secondary gear 205 coupled to tertiary gear 207. In operation, as upper section of positioning guide 171 is translated, spooling mechanism 194 will rotate, causing primary gear 201 to rotate. Secondary gear 205 is in contact with primary gear 201, and will rotate with primary gear 201. Secondary gear 205 is also in contact tertiary gear 207 which is in contact with rack 172a of lower section of positioning guide 172. Thus, through this chain of gears, lower section of positioning guide 172 can have a different rate of travel than that of upper section of positioning guide 171, the rate of travel being dictated by the associated gearing ratio. Accordingly, lower section of positioning guide 172 can be extended at a displacement that is different from that of the displacement of upper section of positioning guide 171.

It should be noted that the terms upper and lower are understood to be relative, and are not to be limiting as to absolute “locations” of the positioning guide's position on the ICD. In some embodiments, the upper/lower sections may be lateral or offset to each other, therefore it is understood that the use of the terms upper and lower are for illustrative purposes.

While the positioning guide gear train is shown with three (3) gears, it is clearly possible to have more (or even less) gears, depending on sizing allowances and operational objectives. For example, FIG. 20's design is tailored to “injecting” material as the cannula 12 is withdrawn. By removing tertiary gear 207 and having secondary gear directly engage rack 172a of lower section of positioning guide 172, a mode of operation for “injection” can occur during insertion of the cannula 12.

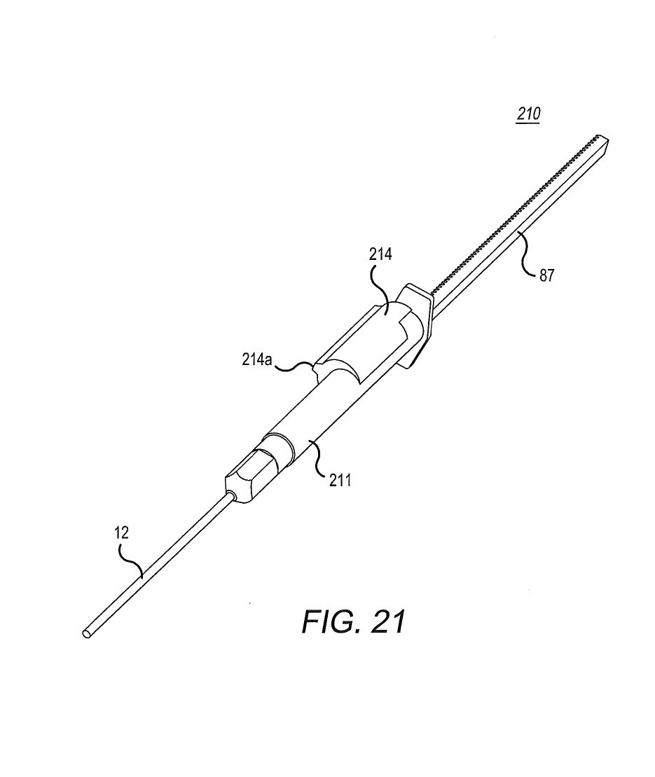

FIG. 21 is an illustration 210 of a syringe adapter for use with some embodiments of the injection control devices. The syringe adapter 214 operates as a means to accommodate smaller sized syringes 211 (for example, 3 cc syringes) into an ICD that is designed for larger syringes. Syringe adapter 214 can be a removable, truncated semi-circular clip that “clips” onto the body of smaller syringe 211 with an optional guiding ridge 214a, to center the syringe 211 into the ICD. Accommodating a smaller syringe can allow the practitioner to reduce the amount of injectate extruded from the tip of the cannula per unit distance that the cannula is withdrawn from the subject, thus effectively changing the gear ratio. Of course, it is understood that other means or devices that functionally increase the diameter of the syringe body for matching to the ICD may be utilized without departing from the spirit and scope of this description.

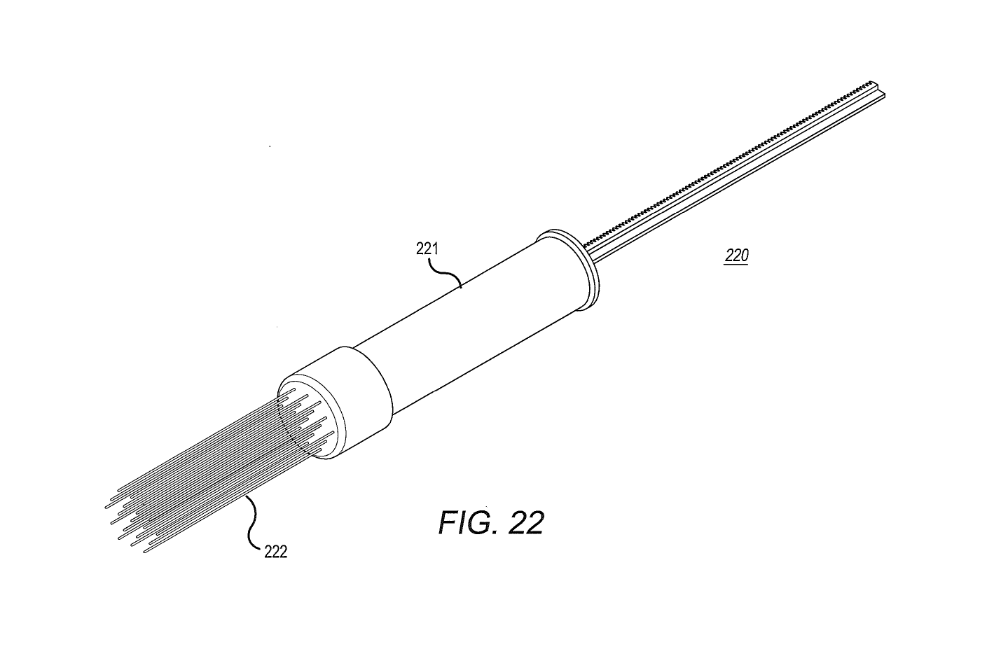

FIG. 22 is an illustration 220 of a multi-cannula syringe for use with some embodiments of the injection control devices. A multi-cannula 222 syringe 221 can be arranged in a grid pattern, for example, incorporated into a single hub that is attached to syringe 221. Using a grid pattern allows for dispersion/extraction of material within a region, eliminating the need for multiple single passes.

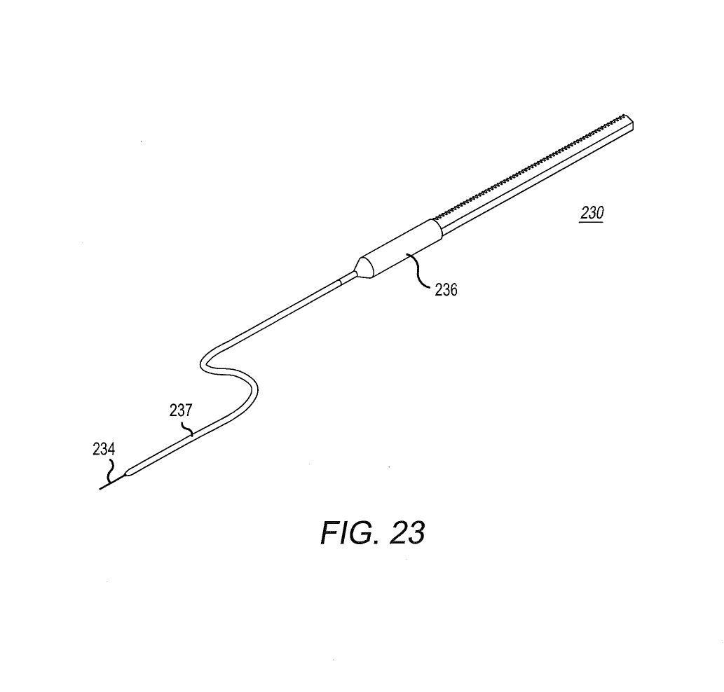

FIG. 23 is an illustration 230 of a flexible-cannula syringe for use with some embodiments of the injection control devices. Cannula 234 may be of a flexible nature 237 (catheter-like) so that the target zone can be through non-linear channels such as arteries, veins, or around obstacles. Syringe body 236 would be fixed in the injection control device with syringe rack 232b. Cannula 234 could be retractable so that intravascular insertion could be achieved. For example, cannula 234 could be inserted such that it abuts the surface to be injected (e.g., myocardium) and establishes the relative position for treatment.

In some embodiments, the outer flexible cannula 237 can function as the positioning guide linearly extensible from the body of the device. A sharp needle or blunt cannula 234 is contained within the outer flexible cannula 237 and connected with the syringe 236 via a smaller flexible inner cannula (not shown) contained within the outer cannula 237. The combined flexible cannula could be inserted through a lumen such as the alimentary tract or femoral artery with the outer cannula (positioning guide) 237 in an extended position such that the needle/cannula 234 at the tip of the inner cannula is sheathed, protecting the tissues. The end of the outer cannula (positioning guide) 237 is then passed and abutted against the site to be injected. The contained needle/cannula and the attached flexible inner cannula is advanced relative to the outer cannula 237 such that the needle/cannula 234 is advanced into the target tissue. The needle/cannula 234 is then withdrawn relative to the outer flexible cannula (positioning guide) 237. This in turn activates the gear mechanism causing the deposition of injectate at a rate that is linearly proportional to the rate of withdrawal of the needle/cannula 234 from the target tissue. This design is intended to facilitate the use of the ICD through long and possibly non-linear channels such as the alimentary, respiratory, and urinary tracts in addition to the cardiovascular system. This embodiment will allow for other endoscopic, endovascular and laparoscopic applications also.

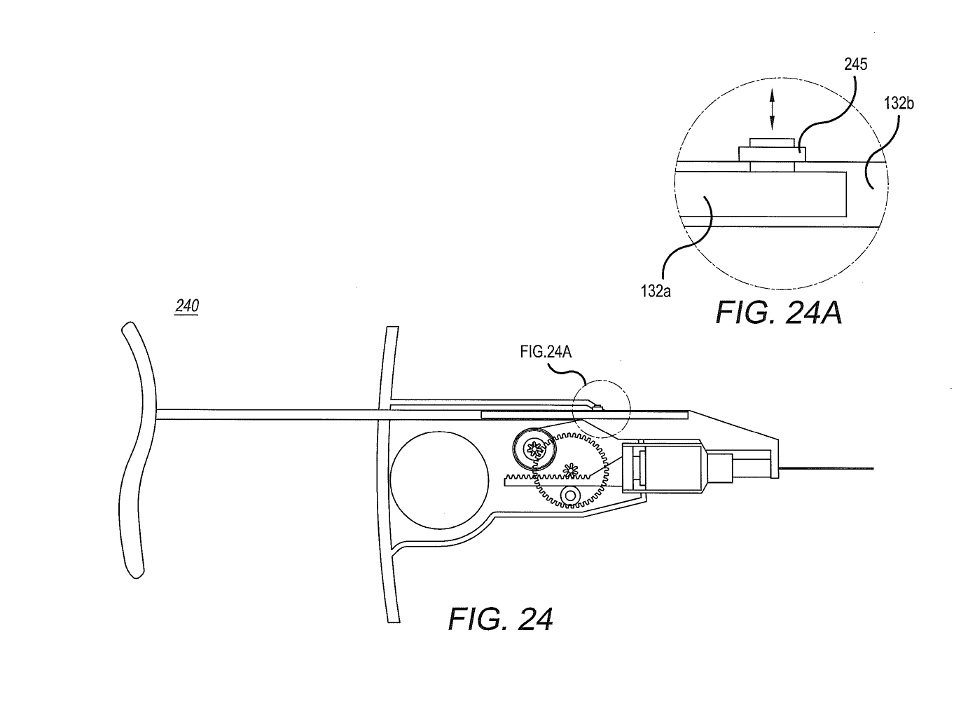

FIG. 24 is an illustration 240 of a cut-away view of a modified embodiment of an injection control device with a disengagable positioning guide. This illustration is a modification of the fifth embodiment/configuration shown in FIG. 15. The contiguous positioning guide 132 of FIG. 15 is substituted with a positioning guide (FIG. 24) that is “slidable” within itself so as to allow the user to compress/push on the positioning guide's handle-side end while allowing the positioning guides' cannula-side end to remain stationary. In other words, in some embodiments, the injection device may be configured so as to allow the positioning guide to be disengaged from the gear mechanism, permitting injection of material or withdrawal of material to occur with the cannula in a stationary position. This allows the practitioner the option to “stop” the proportional administration and inject more material (or withdraw more material) for a given position of the cannula.

The example shown in FIG. 24 is a sleeve configuration enabling section 132a to advance or retract freely within the sleeve 132b, thus allowing injection to occur while the tip of the cannula is stationary and the rate of injection is not proportional to the travel of the cannula. It is understood that other forms of allowing motion of one section to proceed while fixing motion of another section, of a non-sliding nature, are possible. To allow the engagement/disengagement, a locking mechanism 245 is shown in FIG. 24A, a blowup of the corresponding section on FIG. 24. The locking mechanism 245 can simply be a friction causing pushbutton that binds the outer 132b section to the inner 132a section. While a pushbutton mechanism is illustrated, it is well known that other forms of engagement/disengagement are known in the art and may be utilized without departing from the spirit and scope of this disclosure.

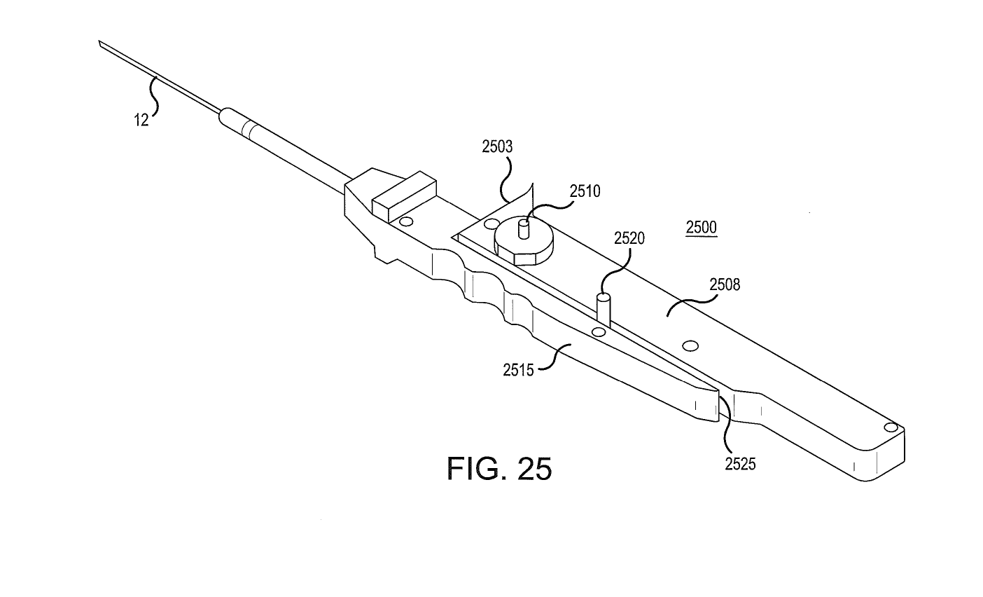

FIG. 25 is an illustration 2500 of a perspective view of the main body of a modified embodiment of an injection control device configured for independent plunger movement, with the positioning guide removed from view. As a point of reference, the “body” configuration is similar to the embodiment shown in FIG. 12 with several as-noted exceptions. Clutch button 2510 is shown adjacent to thumb rest/safety ridge 2503, and operates as an engagement/disengagement mechanism for the clutch (not shown) that allows (when clutch is disengaged) independent movement of the plunger retraction arm 2520, without activating the positioning guide (not shown). Plunger retraction arm 2520 is coupled either directly or indirectly to the plunger rack (not shown) and travels through 2525 in ICD body 2508 or in finger grip section 2515 of the ICD body 2508. The slot 2525 may be a general opening or constitute an exposed part of the body 2508 for movement of the plunger retraction arm 2520. The plunger retraction arm 2520 permits the practitioner to physically control the syringe plunger (not shown) and bypass the syringe plunger-to-gearing mechanism in the ICD. In some embodiments, finger grip section 2515 can be removed from the ICD body 2508 to provide access to the syringe body and internal gearing/mechanisms (not shown).

This embodiment enables the user of the ICD to release (via engagement of the clutch button 2510) the clutch from actively coupling the main gearing of the ICD and associated syringe plunger or plunger rack. Thus, allowing the practitioner to retract the syringe plunger attached to plunger retraction arm 2520 and refill the syringe without removal of the syringe from the ICD. This also allows the syringe to be aspirated to assess the possible intravascular position of the tip of the cannula 12. This latter capability is a significant consideration when using the ICD for fillers (for example, synthetic) with sharp cannulas 12. This embodiment also allows the user to manually engage the plunger rack/plunger retraction arm 2520, to allow for an override of the proportional injection/extraction of material capability of the ICD. This may be particularly helpful in circumstances where the practitioner wishes to add/remove more than originally planned, or to skip a section of the area to be treated. For example, in the latter case, the practitioner may which to place/remove filler (or other material) in a dash-like track, having one section with proportional filler and skipping a section and then having the next section with proportional material, etc. Or, adjusting on the fly, the amount to be delivered/removed by engaging the clutch button 2510.

Of course, having the ability to reload the syringe in mid-operation, without removing the ICD from the patient is another possible feature. To access the syringe body (or interior, thereof) in mid-operation is especially useful when using the ICD to aspirate/withdraw material from a patient in the course of a long “track” and the syringe body becomes full (or empty) during mid-track. The clutch disengagement feature allows the syringe contents to be handled without removing the syringe from the body of the device, saving significant operation time. As one example of an implementation of this embodiment, a rigid “T” connector can be used to connect the syringe with the cannula. The straight through portion of the connector and the side port can contain one-way valves whose purpose is to allow only filling of the syringe as the plunger is withdrawn and only injection of the filler through the cannula as the plunger is depressed (presuming this is the mode of operation desired, otherwise the reverse can be accommodated). The side port remains connected via a tubing to a large reservoir. This system allows rapid refilling of the syringe while ensuring the harvesting and injection of materials through a completely closed system.

As another example, using a canister (or other similar) system in the syringe body, the canister can be removed during mid-track without removing the ICD, thus allowing the patient to only endure a single puncture hole per multiple canister procedure. This capability is further exemplified if a multiple cannula syringe is utilized, as the increase in cannulas will cause a greater amount of material to be extracted/injected per cannula travel distance.

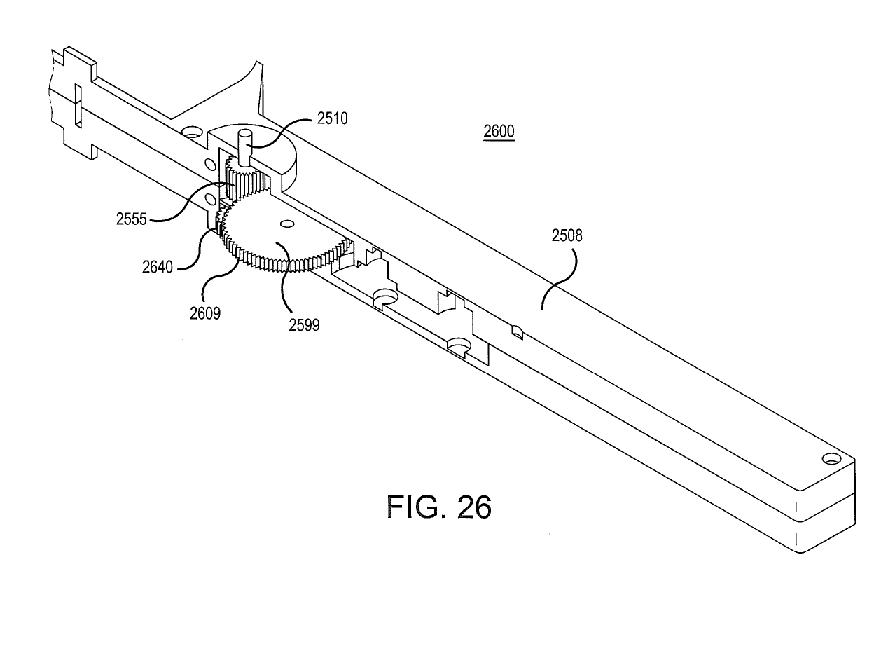

FIG. 26 is another illustration 2600 of the embodiment of FIG. 25, but with the finger grip section 2515 removed, exposing the syringe rack/main gear 2599, clutch-to-main gear 2555 and plunger gear 2640. In some embodiments, the opposite section of the body 2508 may be removed for access to the syringe body, depending on design preference. Clutch button 2510 is shown as part of the axle to clutch-to-main gear 2555. In this embodiment, clutch button 2510 as part of the axle is rigidly attached to clutch-to-main gear 2555 and slides “into” and “out of” the body 2508 to cause clutch-to-main gear 2555 to laterally (non-rotationally) slide across teeth 2609 of syringe rack/main gear 2599. In some embodiments, the clutch button 2510 may be directly or indirectly connected to the axle of clutch-to-main gear 2555. That is, the axle portion may be wholly interior to the body 2508 and the clutch button 2510 may be the exposed, triggerable mechanism. In other embodiments, the clutch button 2510 may not be “in-line”with the axle but in the form of a flip trigger, as in the safety switch seen in hand guns. Accordingly, alternative triggering schemes may be utilized without departing from the spirit and scope of this disclosure.

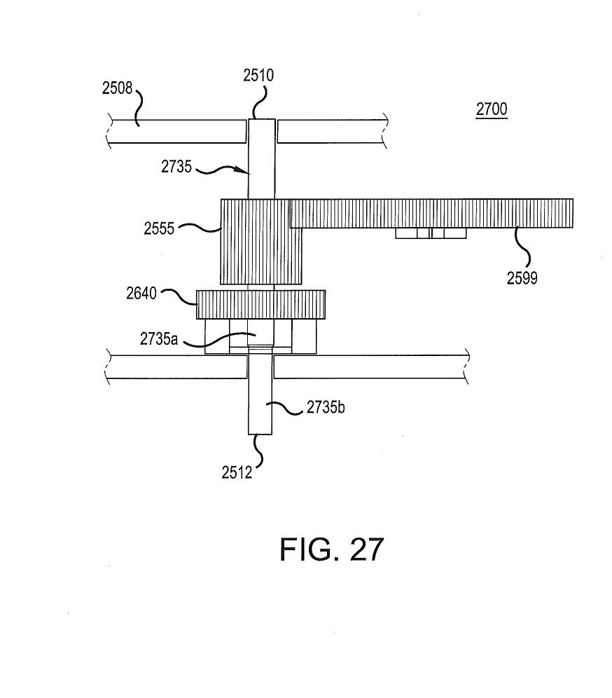

FIG. 27 is a top view illustration 2700 of a clutch assembly in a engaged position. This embodiment shows clutch button 2510 as pushed into body 2508, causing clutch-to-main gear 2555 to be displaced across syringe rack/main gear 2599, placing clutch-to-main gear 2555 adjacent but not in contact with plunger gear 2640. Clutch-to-main gear 2555 is the only gear in this embodiment that is laterally moveable, being attached to clutch button 2510 via axle 2735. Interaction of clutch-to-main gear 2555 with plunger gear 2640 is accomplished with axle 2735 which is composed of at least two different diameters being sized so that the larger diameter 2735a engages the interior of plunger gear 2640 and the smaller diameter 2735b does not engage the interior of plunger gear 2640. Plunger gear 2640 interior is gapped to allow axle 2735 to slide into and out of plunger gear 2640, but sized to permit engagement (e.g., rotation) of the plunger gear 2640 when the larger diameter 2735a is inside of plunger gear 2640 and non-engagement (e.g., no rotation) of the plunger gear 2640 when the smaller diameter 2735b is inside plunger gear 2640.

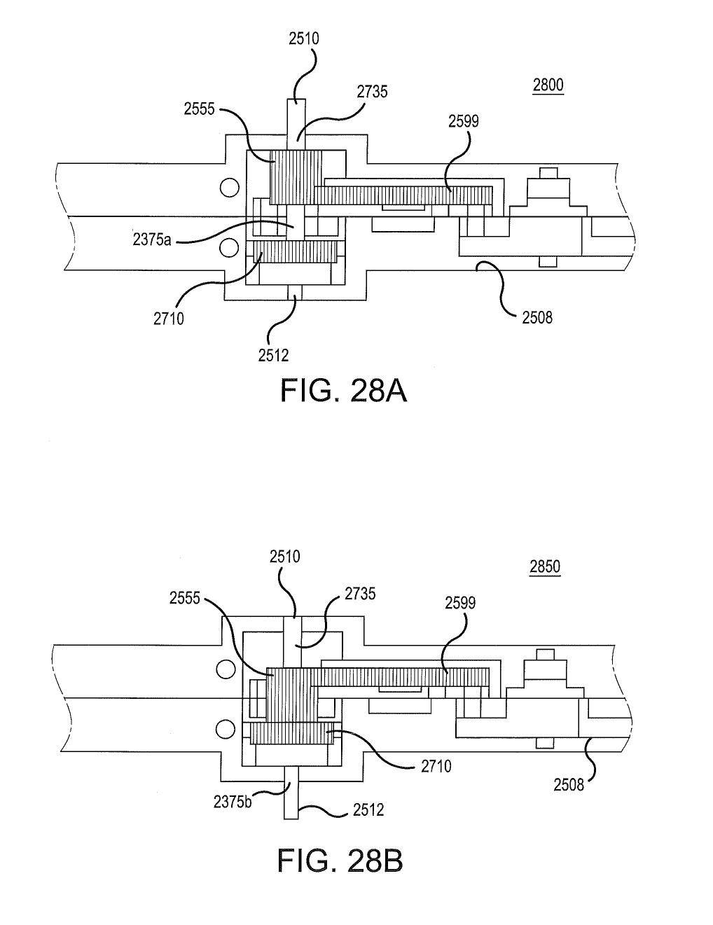

FIGS. 28A-B are closeup illustrations of a disengaged mode 2800 and engaged mode 2850 of the clutch assembly. FIG. 28A shows clutch button 2510 pushed “into” body 2508 to cause axle 2735 (having different sized diameters proximal to plunger gear 2640) to move the larger axle diameter 2735a into plunger gear 2640 to engage it. FIG. 28B shows clutch button 2510 pushed “out of” body 2508 (or pushed from the other side 2512) will cause the larger axle diameter 2735a out of the plunger gear 2640, being replaced with non-engaging smaller axle diameter 2735b to disengage the disengage it.

Of course, in some embodiments, it may be desirable to reverse the larger/smaller order of the axle 2735 to cause disengagement either though pushing in or pushing out clutch button 2510. It should also be appreciated that while the above embodiments illustrate the clutch engaging/disengaging via interaction from a multiply-sized axle arrangement, other procedures, mechanisms for engaging/disengaging may be utilized without departing from the spirit and scope of this disclosure. For example, raised splines may be used to cause engagement, or the clutch-to-main gear 2555 may be configured to have its teeth “slide” into/out of the gears of plunger gear 2640, rather than relying on the axle 2735 for contact. It may also be possible to have a pin on the side of clutch-to-main gear engage with plunger gear 2640, rather than via the axle 2735.

As another one of many possible examples, clutch-to-main gear 2555 can be of a standard width or thickness that engages with either with a hexagonal (or angled/non-uniform circumference shape) axle 2735 but with a loose fit to allow lateral movement relative to the clutch-to-main gear 2555 but not allow rotational movement relative to clutch-to-main gear 2555. This would allow clutch-to-main gear 2555 to remain stationary with respect to syringe rack/main gear 2599 and constantly be engaged to the axle 2735 from a rotational perspective as the axle 2735 is mobilized laterally to engage or disengage the two diameters with the clutch within plunger gear 2640. Bearing or other mechanical elements may be also used to bring about the above functionalities.

Therefore, the approaches shown should not be interpreted as limiting but illustrative of many possible ways to allow the clutch to engage/disengage with the plunger gear 2640, understanding that one of ordinary skill in the art may contrive alternative schemes for “engagement” and “disengagement” without departing from the spirit and scope of this disclosure.

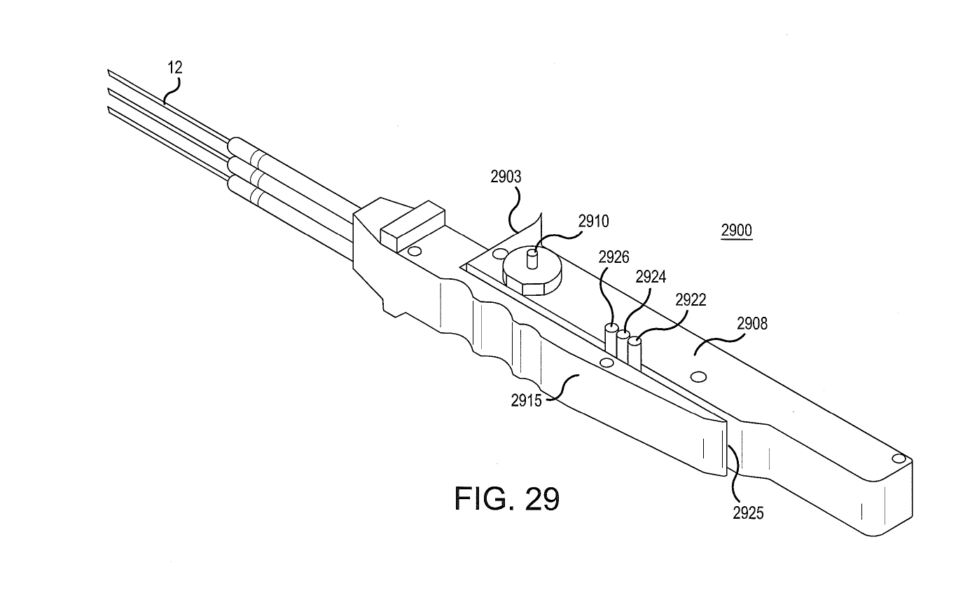

FIG. 29 is an illustration 2900 of a multi-syringe configuration of an ICD with clutch button 2910 in proximity to thumb rest/safety ridge 2903. This embodiment contemplates the possibility, that in a multi-cannula ICD (for example, FIG. 22), an occasion may occur where the subject material may not travel equally though the cannulas (a cannula either being restricted in some way as compared to the other cannulas, such as a particle in the filler, or body fluid/material, etc.), resulting in a differential pressure between the cannulas to bring rise to the material being unequally injected/aspirated between the cannulas.

In FIG. 29, the use of multiple syringes, each operating with a dedicated plunger assures direct pressure/suction on each cannula to minimize this occurrence. The embodiment of FIG. 29 is illustrated with the capability for independent operation of the plunger retraction arms 2922, 2924, 2926 associated with the respective syringes, traveling along slot 2925 in ICD body 2908. Of course, the plunger refraction arms 2922, 2924, 2926 may be fixed to each other (or represented by a single plunger retraction arm) to operate in unison, if so desired. Therefore, an express embodiment having multiple syringes with their respective plungers “connected” to give equal pressure across the syringes is contemplated. Further, another express embodiment having multiple syringes, but without independently retractable plunger(s), is also contemplated, wherein the ICD body would be similar to those shown in FIGS. 1, 12 and so forth, but configured for multiple syringes.

In an embodiment with independent plunger operation, the clutch button 2910 can be configured to individually engage/disengage with the internal mechanisms, for example, with an axle region having an appropriately sized diameter that is matched for the respective syringe/plunger arm as the clutch button 2910 is pressed in or out. Some form of positive feedback, such as a click or color-coding (to signify the engaged syringe) on clutch button 2910 can be facilitated.

As stated above, alternative schemes for engaging the respective (or all syringes) may be contemplated without departing from the spirit and scope of this disclosure. For example, a rotating versus push/pull scheme may be used in the clutch mechanism. That is, a knob that rotates or a toggle switch can be configured to perform the equivalent or similar operations described above.

While the embodiments of the injection control device are shown in the above FIGS. as requiring manual movement to effect the travel of the filler material, it should become apparent, based on this disclosure, that automatic movement may be effected by a motor. Thus, the linkage between the various parts may be substituted by a motor or electromechanical device. Similarly, a hydraulic system for controlled the injection rate or suction rate may be implemented without departing from the spirit and scope of this disclosure. By use of an electromechanical device or system, some embodiments of the injection control device may be easily adapted to larger volume operations, such as, breast and buttock augmentation. Additionally, an alternative “gearing” mechanism may be desired, non-limiting examples being springs, spring motor, screw type racks or worm gears, as well as piezoelectric travel engines, and so forth. In one contemplated embodiment, the positioning guide may be telescoping in nature, or flexible so as to “coil” within the body during retraction.

A virtual transmission activating system could be devised, using laser ranging, stereotactic, etc. so that the transmission activating system (i.e., positioning guide) does not physically extend from the body of the ICD. The virtual system would operate as a means to track the position and/or velocity of the body of the ICD and/or cannula relative to the subject, and control the rate of injection/extraction. A computer or computerized system could be utilized to digitally control the stated actions, rather than using simple mechanical means. For example, the virtual transmission activating system could drive a servo to control the syringe plunger at a predetermined rate to that of the body/syringe's motion.

For example, the virtual transmission activating system could comprise a virtual positioning guide that utilizes any means of tracking the position, orientation, direction of travel and speed of the injection control device and/or the tip of its cannula in relation to the subject being injected. Non-limiting examples of stereotactic surgical devices in current use capable of tracking in this manner include Medtronic Fusion image guided surgery system that uses an electromagnetic tracking system and the Stryker® iNtellect surgical navigation system (registered to Stryker Leibinger GmbH & Co K) that uses an optical tacking system.

In various applications, it is envisioned that using a cylindrical cannula will result in cylindrical tracks of material left in the channel created by the cannula's intrusion. Using computer/automated devices, an increased degree of control can be obtained in the amount and “shape” of the deposited material or extracted material as well as variation of the injection/extraction profile. For example, conical, elongated spheres, or series of spheres could be produced. A similar result can also be obtained by using a camming system in the transmission system to periodically delay/increase the rate of injection/extraction. Following this, a robotic system which precisely controls the position and rate of motion of the cannula and/or rate of injection/extraction could be implemented in the ICD. Moreover, while the “applications” are in the context of a cannula “inside” a subject, the ICD can be easily adapted to regulate the rate of extrusion of a fluid for a topical application.

It will be understood that many additional changes in the details, materials, steps and arrangement of parts, which have been herein described and illustrated to explain the nature of the disclosure, may be made by those skilled in the art within the principle and scope of the disclosure as expressed in the appended claims.

What is claimed is:

1. An injection control device (ICD) for a syringe, adapted to inject/extract material into/from a subject at a rate proportional to the rate of movement of the syringe, comprising:
an ICD body with a proximal and distal end, comprising:
a syringe holder positioned at the proximal end, securing at least one accommodated syringe from movement relative to the body;
a reference member opening at the proximal end; and
an arm opening along the body, to accommodate travel of an exposed plunger retraction arm of a plunger of the at least one accommodated syringe;
a transmission reference member extendable outward from the body via the reference member opening;
a clutch assembly coupled to the body with an engagement/disengagement release trigger to allow independent movement of the plunger retraction arm; and
a transmission system coupled to the body, controllably engaged to the clutch, wherein a first power transfer section of the transmission is connected to the transmission reference member and a second power transfer section of the transmission is connected to a plunger of the at least one accommodated syringe, the transmission system configured to translate motion from movement of the body, in relation to a fixed state of the transmission reference member, to action on the plunger of the at least one accommodated syringe,
wherein the clutch is configured to allow the transmission reference member to be positioned, without transferring power to the transmission system,
wherein the plunger action is proportional to the movement of the body, resulting in material being injected/extracted into/from a subject as a cannula of the at least one accommodated syringe is traveling with the movement of the body.

2. The device of claim 1, wherein the release trigger and clutch is a mechanism comprising:
a translatable axle of the transmission system having a first diameter and a second diameter smaller than the first diameter, an end thereof being directly or indirectly engaged via the release trigger;
an axle gear attached to the translatable axle, a teeth of the axle gear contacting a teeth of a main gear of the transmission system, in both an engaged and disengaged state of the release trigger; and
a plunger gear displaced from the axle gear and in alignment with the translatable axle, a center hole of the plunger gear sized larger than the second diameter of the translatable axle and smaller than the first diameter of the translatable axle, so as to cause engagement of the plunger gear with the translatable axle when the first diameter of the translatable axle is translated into the plunger gear hole and to cause disengagement of the plunger gear when the second diameter of the translatable axle is translated into the plunger gear hole,
wherein in an disengaged state, the plunger gear is disengaged from the clutch and transmission system, allowing the plunger to be moved without affecting the first power transfer section of the transmission system.

3. The device of claim 1, wherein the ICD body is a multi-piece body and the arm opening delineates a section of the body that is removable, exposing the at least one accommodated syringe.

4. The device of claim 2, wherein the ICD body is a multi-piece body and the arm opening delineates a section of the body that is removable, exposing the at least one accommodated syringe.

5. The device of claim 1, wherein the second power transfer section of the transmission is indirectly coupled to the plunger of the at least one accommodated syringe.

6. The device of claim 2, wherein the second power transfer section of the transmission is indirectly coupled to the plunger of the at least one accommodated syringe.

7. The device of claim 1, wherein the cannula of the at least one accommodated syringe is at least one of a plurality of cannulas and a flexible cannula.

8. The device of claim 2, wherein the cannula of the at least one accommodated syringe is at least one of a plurality of cannulas and a flexible cannula.

9. The device of claim 1, further comprising a syringe, wherein the syringe contains at least one of fat, stem cells, hyaluronic acid, polymethylmethacrylate, hydroxyapatite, drug, vaccine, botulinum toxin, bone cement, demineralized bone, and hydrogel.

10. The device of claim 2, further comprising a syringe, wherein the syringe contains at least one of fat, stem cells, hyaluronic acid, polymethylmethacrylate, hydroxyapatite, drug, vaccine, botulinum toxin, bone cement, demineralized bone, and hydrogel.

11. The device of claim 1, wherein the transmission reference member is at least one of an optical or laser ranging, electromagnetic ranging, and stereotactic system utilizing a computer-controlled servo to control the plunger.

12. The device of claim 2, wherein the transmission reference member is at least one of an optical or laser ranging, electromagnetic ranging, and stereotactic system utilizing a computer-controlled servo to control the plunger.

13. A method of injecting/extracting material into/from a subject at a rate controllably proportional to the rate of movement of a syringe attached to an injection control device (ICD), the device comprising:
placing an ICD with at least one accommodated syringe's cannula upon or into a subject's tissue, wherein the ICD comprises:
an ICD body with a proximal and distal end, comprising:
a syringe holder positioned at the proximal end, securing the at least one accommodated syringe from movement relative to the body;
a reference member opening at the proximal end; and
an arm opening along the body, to accommodate travel of an exposed plunger retraction arm of a plunger of the at least one accommodated syringe;
a transmission reference member extendable outward from the body via the reference member opening;
a clutch assembly coupled to the body with an engagement/disengagement release trigger to allow independent movement of the plunger retraction arm; and
a transmission system coupled to the body, controllably engaged to the clutch, wherein a first power transfer section of the transmission is connected to the transmission reference member and a second power transfer section of the transmission is connected to a plunger of the accommodated syringe, the transmission system configured to translate motion from movement of the body, in relation to a fixed state of the transmission reference member, to action on the plunger of the at least one accommodated syringe,
wherein the clutch is configured to allow the transmission reference member to be positioned, without transferring power to the transmission system, positioning the transmission reference member; and
pressing on the transmission reference member and withdrawing or advancing the body of the ICD,
wherein the plunger action is proportional to the movement of the body, resulting in material being injected/extracted into/from a subject as a cannula of the at least one accommodated syringe is traveling with the movement of the body.

14. The method of claim 13, wherein the release trigger and clutch is a mechanism comprising:
a translatable axle of the transmission system having a first diameter and a second diameter smaller than the first diameter, an end thereof being directly or indirectly engaged via the release trigger;
an axle gear attached to the translatable axle, a teeth of the axle gear contacting a teeth of a main gear of the transmission system, in both an engaged and disengaged state of the release trigger; and
a plunger gear displaced from the axle gear and in alignment with the translatable axle, a center hole of the plunger gear sized larger than the second diameter of the translatable axle and smaller than the first diameter of the translatable axle, so as to cause engagement of the plunger gear with the translatable axle when the first diameter of the translatable axle is translated into the plunger gear hole and to cause disengagement of the plunger gear when the second diameter of the translatable axle is translated into the plunger gear hole,
wherein in an disengaged state, the plunger gear is disengaged from the clutch and transmission system, allowing the plunger to be moved without affecting the first power transfer section of the transmission system.

15. The method of claim 13, wherein the release trigger is in an disengaged state.

16. The method of claim 13, wherein the cannula of the at least one accommodated syringe is at least one of a plurality of cannulas and a flexible cannula, and the device is used for at least one of a endoscopic, endovascular and laparoscopic procedure.

17. An injection control device (ICD) for a syringe, adapted to inject/extract material into/from a subject at a rate proportional to the rate of movement of the syringe, comprising:
an ICD body with a proximal and distal end, comprising:
a syringe holder positioned at the proximal end, securing at least one accommodated syringe from movement relative to in the body;
a reference member opening at the proximal end; and
means for accommodating travel of an exposed plunger retraction arm of a plunger of the at least one accommodated syringe;
a reference member extendable outward from the body via the reference member opening;
a means for clutching coupled to the body with a triggering means to allow independent movement of the plunger retraction arm; and
a means for redirecting motion coupled to the body, controllably engaged to the means for clutching, wherein a first power transfer section of the means for redirecting motion is connected to the reference member and a second power transfer section of the means for redirecting motion is connected to a plunger of the at least one accommodated syringe, the means for redirecting motion configured to translate motion from movement of the body, in relation to a fixed state of the reference member, to action on the plunger of the at least one accommodated syringe,
wherein the means for clutching is configured to allow the reference member to be positioned, without transferring power to the means for redirecting motion,
wherein the plunger action is proportional to the movement of the body, resulting in material being injected/extracted into/from a subject as a cannula of the at least one accommodated syringe is traveling with the movement of the body.

18. The device of claim 17, wherein the ICD body is a multi-piece body and the means for accommodating travel delineates a section of the body that is removable, exposing the at least one accommodated syringe.

19. The device of claim 17, wherein the cannula of the at least one accommodated syringe is at least one of a plurality of cannulas and a flexible cannula.

20. The device of claim 17, further comprising a syringe, wherein the syringe contains at least one of fat, stem cells, hyaluronic acid, polymethylmethacrylate, hydroxyapatite, drug, vaccine, botulinum toxin, bone cement, demineralized bone, and hydrogel
